(19) United States
(12) Patent Application Publication
(10) Pub. No.: US 2026/0124193 A1
(43) Pub. Date: May 7, 2026

(54) PHARMACEUTICAL COMPOSITION FOR THE TREATMENT OF RADIATION INDUCED TOXICITY

(71) Applicant: Zydus Lifesciences Limited, Ahmedabad (IN)

(72) Inventors: Mukul Jain, Ahmedabad (IN); Amit Joharapurkar, Ahmedabad (IN); Vishal Patel, Ahmedabad (IN); Jay Kothari, Ahmedabad (IN); Muthaiyyan Essakimuthu Kannan, Ahmedabad (IN); Ritu Laddha, Ahmedabad (IN); Mukesh Ukawala, Ahmedabad (IN); Sunil Sahu, Ahmedabad (IN)

(21) Appl. No.: 19/434,846

(22) Filed: Dec. 29, 2025

Related U.S. Application Data
(63) Continuation of application No. PCT/IN2025/051488, filed on Sep. 12, 2025.

(30) Foreign Application Priority Data
Sep. 13, 2024 (IN) 202421069288
Sep. 27, 2024 (IN) 202421073122

(51) Int. Cl. A61K 31/4704; A61K 9/00; A61K 9/08; A61K 38/19; A61K 47/02; A61K 47/08; A61K 47/60; A61K 47/68; A61P 7/00; A61P 39/00

(52) U.S. Cl. CPC A61K 31/4704; A61K 9/0019; A61K 9/08; A61K 38/193; A61K 38/196; A61K 47/02; A61K 47/08; A61K 47/60; A61K 47/6813; A61P 7/00; A61P 39/00

(57) ABSTRACT

The present invention relates to parenteral pharmaceutical compositions of suitable Hypoxia-Inducible Factor Prolyl Hydroxylase Inhibitor, particularly to Desidustat or suitable pharmaceutically acceptable salts thereof. The invention also relates to processes for preparing such compositions. The present invention also relates to the development of therapeutic compound for the treatment of radiation induced toxicity. Specifically, present invention relates to use of Desidustat or its pharmaceutically acceptable salt or oral and parenteral pharmaceutical composition thereof for the treatment of radiation induced toxicity.

### CROSS-REFERENCE TO RELATED APPLICATIONS

This application is a continuation of International Application No. PCT/IN2025/051488, filed on Sep. 12, 2025, which application claims the benefit of, and priority to, IN Application No. 202421069288, filed Sep. 13, 2024 and IN Application No. 202421073122, filed Sep. 27, 2024, the contents of which are hereby incorporated by reference herein in their entirety.

### FIELD OF THE INVENTION

The present invention relates to parenteral pharmaceutical compositions of suitable Hypoxia-Inducible Factor Prolyl Hydroxylase Inhibitor, particularly to Desidustat or a pharmaceutically acceptable salt thereof. The invention also relates to processes for preparing such compositions. The present invention also relates to the development of therapeutic compound for the treatment of radiation induced toxicity. Specifically, present invention relates to use of Desidustat or its pharmaceutically acceptable salt or oral and parenteral pharmaceutical composition thereof for the treatment of radiation induced toxicity.

### BACKGROUND OF THE INVENTION

Desidustat is a novel small-molecule drug for the treatment of anemia associated with chronic kidney disease (CKD). It functions as a hypoxia-inducible factor prolyl hydroxylase (HIF-PH) inhibitor, which stabilizes HIF-α and stimulates endogenous erythropoietin production, thereby promoting red blood cell formation and improving iron metabolism. Structurally, Desidustat is a quinolinone derivative with the chemical formula C₁₆Hi₆N₂O₆. Desidustat was first approved in India in 2022 and has undergone extensive clinical evaluation, including comparative studies with erythropoietin analogs. Desidustat was disclosed in WO2014102818. WO2021186356 focuses on optimized pharmaceutical compositions, particularly oral formulation. There is no parenteral composition available for Desidustat. Despite the advances that have been made to date, there is still an ongoing need for different dosage forms of Desidustat that can be used for treating various medical disorders.

### SUMMARY OF THE INVENTION

The present invention relates to parenteral pharmaceutical compositions of suitable the prolyl hydroxylase inhibitors, particularly to Desidustat or suitable pharmaceutically acceptable salts thereof. The invention also relates to processes for preparing such compositions. The present invention also relates to the development of therapeutic compound for the treatment of radiation induced toxicity. Specifically, present invention relates to use of Desidustat or its pharmaceutically acceptable salt or oral and parenteral pharmaceutical composition thereof for the treatment of radiation induced toxicity.

The structure of Desidustat is set forth below:

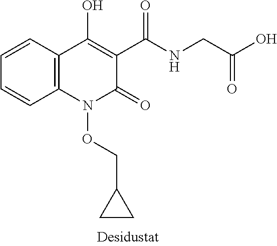 [SMILES: O=C(O)CNC(=O)c1c(O)c2ccccc2n(OCC2CC2)c1=O]

Parenteral drug formulations have become a very important component in the arsenal of available drug delivery options, particularly for drugs having effect on anemia. Parenteral routes of administration, including subcutaneous, intramuscular and intravenous injection, offer numerous benefits over oral delivery in particular situations, for a wide variety of drugs. For example, parenteral administration of a drug typically results in attainment of a therapeutically effective blood serum concentration of the drug in a shorter time than is achievable by oral administration. This is especially true of intravenous injection, whereby the drug is placed directly in the bloodstream. Parenteral administration can also result in more predictable blood serum concentrations of a drug, because losses in the gastrointestinal tract due to metabolism, binding to food and other causes are eliminated. For similar reasons, parenteral administration often permits dose reduction. Parenteral administration is generally the preferred method of drug delivery in emergency situations and is also useful in treating subjects who are uncooperative, unconscious, or otherwise unable or unwilling to accept oral medication. Thus, due to above listed benefits scientists of the present invention have developed parenteral composition of Desidustat.

Radiation therapy or acute radiation syndrome or radiation sickness caused by exposure to high amounts of ionizing radiation in a short period of time or prolong exposure. Depending upon the amount and time of exposure the effect of radiation may last for several days to months or years. If exposure is high and for prolong period of time, it may damage specific organ systems and that can lead to death within hours or months [1]. Radiation mainly affects hematopoietic, gastrointestinal, and neurovascular systems. Radiation therapy used in cancer also affects these systems. Radiation mostly affects the rapidly dividing cell and causes irreparable damage to DNA. Chemotherapeutic agents such as cisplatin, melphalan and cyclophosphamide also causes indirect bone marrow cell damage by preventing DNA replication. Current treatment of radiation exposure includes blood transfusions, antibiotics, colony-stimulating factors, or stem cell transplant. Filgrastim, sargramostim and romiplostim are the colony stimulating factor approved for acute radiation syndrome [2]. Anemia is the most common symptoms of radiation exposure that affects the quality of life. Hypoxia-inducible factors (HIF) regulate oxygen level, regulated by prolyl hydroxylase domain (PHD) enzymes [3]. Inhibition of PHD can stabilize HIFs thus increasing the availability of HIFs. Stabilization of HIFs increases erythropoietin (EPO) secretion by PHD inhibitors thus increases erythroid progenitor maturation in the bone marrow and elevates anemia. Stabilization of HIF also exerts anti-inflammatory effect and prevents oxidative stress [4-5]. Desidustat is a PHD inhibitor approved for the treatment of chronic kidney disease. It alleviates anemia, suppress inflammation and suppress oxidative stress markers [6-7]. Thus, Desidustat may be used to in the treatment of radiation sickness to increases hematopoietic deficiencies, reduce inflammation, and suppress oxidative stress. The Desidustat may be combined with other colony stimulating factors or stem cell transplantation in the treatment of radiation exposure. Several other prolyl hydroxylase inhibitors have been disclosed in EP661269, WO2007070359, WO2008076425, WO2011007856, WO2012106472, and WO2013043621. Specifically, WO2004108681 and WO2008002576 covers the prolyl hydroxylase inhibitors named Roxadustat and Vadadustat respectively.

Anemia, particularly iron-deficiency anemia, is primarily caused by insufficient availability of iron, which is a critical component of hemoglobin synthesis. This form of anemia typically results from chronic blood loss, inadequate dietary intake, or malabsorption syndromes. The bone marrow remains functionally competent but is unable to produce adequate hemoglobin-rich erythrocytes due to the lack of essential substrates whereas radiation-induced anemia arises as a consequence of exposure to ionizing radiation, which exerts cytotoxic effects on the hematopoietic stem and progenitor cells within the bone marrow. This form of anemia is frequently observed in patients undergoing radiotherapy or exposed to environmental radiation. The damage is not limited to erythropoiesis but may extend to other hematopoietic lineages, resulting in pancytopenia.

Understanding the mechanistic divergence between these two forms of anemia is critical for the development of targeted therapeutic interventions. The invention described herein addresses the unique challenges posed by radiation-induced hematopoietic toxicity and proposes a novel approach for mitigating bone marrow suppression and restoring erythropoiesis.

Radiation-induced toxicities encompass all negative effects from radiation exposure, which can be acute or long-term, on various organs and tissues. Hematopoietic Acute Radiation Syndrome (H-ARS) is one of the three main acute syndromes that occur after a high-dose, short-term radiation exposure, alongside the Gastrointestinal (GI) and Cerebrovascular (CNS) syndromes.

It has surprisingly now been found that Desidustat and its suitable pharmaceutically acceptable salts are effective in the treatment of radiation induced toxicity.

### Embodiments of the Invention

In an embodiment, the present invention provides a parenteral pharmaceutical composition comprises Desidustat or pharmaceutically acceptable salts thereof.

In another embodiment, the present invention provides a parenteral pharmaceutical composition comprises Desidustat or pharmaceutically acceptable salts thereof and suitable pharmaceutically acceptable excipients.

In another embodiment, the present invention provides a parenteral pharmaceutical composition comprises Desidustat or pharmaceutically acceptable salts thereof, suitable alkalizing agent and a suitable pharmaceutically acceptable excipient.

In yet another embodiment, the present invention provides a process for the preparation of suitable parenteral composition comprises Desidustat or pharmaceutically acceptable salts thereof.

In an embodiment, the present invention provides a method of treating or preventing a subject suffering from radiation induced toxicity using Desidustat or pharmaceutically acceptable salts thereof.

In an embodiment, the present invention provides a method of treating or preventing a subject suffering from radiation induced toxicity using Desidustat or pharmaceutically acceptable salts thereof wherein radiation induced toxicity include Hematopoietic Acute Radiation Syndrome (H-ARS).

In an embodiment, the present invention provides a method of treating or preventing a subject suffering from radiation induced toxicity using parenteral composition of Desidustat or pharmaceutically acceptable salts thereof.

In an embodiment, the present invention provides a method of treating or preventing a subject suffering from radiation induced toxicity using parenteral composition of Desidustat or pharmaceutically acceptable salts thereof wherein radiation induced toxicity includes Hematopoietic Acute Radiation Syndrome (H-ARS).

In an embodiment, the present invention provides a method of treating or preventing a subject suffering from radiation induced toxicity using oral composition of Desidustat or pharmaceutically acceptable salts thereof.

In an embodiment, the present invention provides a method of treating or preventing a subject suffering from radiation induced toxicity using oral composition of Desidustat or pharmaceutically acceptable salts thereof wherein radiation induced toxicity includes Hematopoietic Acute Radiation Syndrome (H-ARS).

In another embodiment, the present invention provides a use of Desidustat or pharmaceutically acceptable salts thereof for the treatment of radiation induced toxicity.

In another embodiment, the present invention provides a use of Desidustat or pharmaceutically acceptable salts thereof for the treatment of radiation induced toxicity wherein radiation induced toxicity include Hematopoietic Acute Radiation Syndrome (H-ARS).

In another embodiment, the present invention provides a use of parenteral pharmaceutical compositions comprises Desidustat or pharmaceutically acceptable salts thereof for the treatment of radiation induced toxicity.

In another embodiment, the present invention provides a use of parenteral pharmaceutical compositions comprises Desidustat or pharmaceutically acceptable salts thereof for the treatment of radiation induced toxicity wherein radiation induced toxicity include Hematopoietic Acute Radiation Syndrome (H-ARS).

In another embodiment, the present invention provides a use of oral pharmaceutical compositions comprises Desidustat or pharmaceutically acceptable salts thereof for the treatment of radiation induced toxicity.

In another embodiment, the present invention provides a use of oral pharmaceutical compositions comprises Desidustat or pharmaceutically acceptable salts thereof for the treatment of radiation induced toxicity wherein radiation induced toxicity include Hematopoietic Acute Radiation Syndrome (H-ARS).

In yet another embodiment the present invention provides the administration of Desidustat or pharmaceutically acceptable salts thereof alone or in combination with other suitable agents as therapeutic agent for the treatment of radiation induced toxicity.

In another embodiment, the present invention provides a parenteral pharmaceutical composition comprises Roxadustat or pharmaceutically acceptable salts thereof and suitable pharmaceutically acceptable excipients.

### DESCRIPTION OF THE FIGURES

FIG. 1. The effect of Desidustat on radiation induced toxicity in C57 mice.

FIG. 2: The effect of Desidustat on cyclophosphamide-induced toxicity in C57 mice.

### DESCRIPTION OF THE INVENTION

The term “Treating” or “treatment” or condition as used herein means: preventing or delaying the appearance of clinical symptoms of the state, disorder or condition developing in a mammal and also include treating or preventing the disease condition.

The term “preventing” refers to barring a subject from acquiring a disorder or disease in the first place.

Term “subject” used herein anywhere in specification means mammals, that include a human, an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

Term “pharmaceutically acceptable” used herein anywhere in the specification means it is acceptable to use for both humans and animals.

Term “Baseline” used herein anywhere in the specification refers to the initial hemoglobin concentration measured in a subject prior to any therapeutic, diagnostic, or experimental intervention. This value is used to assess deviations, treatment efficacy, or physiological changes over time.

The term “USP” means United States Pharmacopeia.

The IUPAC name of Desidustat is (1-(cyclopropylmethoxy)-4-hydroxy-2-oxo-1,2-dihydroquinoline-3-carbonyl)glycine.

Parenteral Pharmaceutical Compositions of Desidustat or their Suitable Pharmaceutically Acceptable Salts

In a further embodiment, the present invention provides a parenteral pharmaceutical composition comprises Desidustat or pharmaceutically acceptable salts thereof.

In another embodiment, the present invention provides a parenteral pharmaceutical composition comprises Desidustat or pharmaceutically acceptable salts thereof and suitable pharmaceutically acceptable excipients.

In another embodiment, the present invention provides a parenteral pharmaceutical composition comprises Desidustat or pharmaceutically acceptable salts thereof, suitable an alkalizing agent and a suitable pharmaceutically acceptable excipient.

The suitable pharmaceutically acceptable salts of Desidustat may be selected from suitable inorganic metal salt or an organic amine salt.

In certain embodiments, the inorganic metal salts can be selected from calcium salt, sodium salt, potassium salt, lithium salt, barium salt, strontium salt, magnesium salt, cesium salt, copper salt, cobalt salt, iron salt, manganese salt, lead salt, aluminium salt, cadmium salt, silver salt, and zinc salt, and the like.

In certain embodiments, the organic amine salts, also referred to herein as organic ammonium salts can be selected from methylammonium salt, dimethylammonium salt, ethylammonium salt, diethyl ammonium salt, n-propyl ammonium salt, isopropyl ammonium salt, diisopropyl ammonium salt, N-methyl isopropyl ammonium salt, n-butyl ammonium salt, t-butyl ammonium salt, 2-butammonium salt, 1,2-ethane diammonium salt, N-methylglucammonium salt, N,N,N-trimethyl ethanolammonium hydroxide (choline) salt, tromethammmonium salt, cyclohexylammonium salt, N-methyl cyclohexylammonium salt, guanidinium salt, N-(4-aminobutyl) guanidinium salt, dicyclohexylammonium salt, benzene-methanammonium salt, ethanolammonium salt, diethanolammonium salt, tris-(hydroxymethyl)methylammonium salt, hydroxylammonium salt, methanammonium salt, benzylammonium salt, N-methylbenzylammonium salt, N-ethyl benzylammonium salt, 4-methoxybenzylammonium salt, pyrrolidinium salt, piperidinium salt, piperazinium salt, morpholinium salt, 2-aminopyrimidinium salt, alaninium salt, lysinium salt, argininium salt, histidinium salt, threoninium salt, prolinium salt, glutammonium salt, glycinium salt, 2-thiopheneethanammonium salt, (2S)-3,3-dimethyl-2-butanammonium salt, cyclopentanammonium salt, cycloheptanammonium salt, meglummonium salt, bethammonium salt, dibenzylammonium salt, diphenylammonium salt, α-naphthylammonium salt, O-phenylenediammonium salt, 1,3-Diaminopropammonium salt, (S)-α-naphthylethylammonium salt, (S)-3-methoxyphenylethylammonium salt, (S)-4-methoxyphenylethylammonium salt, (S)-4-chlorophenylethylammonium salt, (S)-4-methylphenylethylammonium salt, cinchoninium salt, cinchonidinium salt, (−)-quininium salt, triethanolammonium salt, imidazolium salt, ethylenediammonium salt, polammonium salt, morpholinium 4-(2-hydroxyethyl) salt, N—N-diethylethanolammonium salt, deammonium salt, hydrabammonium salt, betammonium salt, adamantanammonium salt, L-adamantanmethylammonium salt, tritylammonium salt, glucammonium salt, N-methyl pyrrolidinium salt, ureammonium salt, procammonium salt, metformmonium salt, hexane1-6-diammonium salt, 2-(2-aminoethoxy)ethanammonium salt, N-methylmorpholinium salt, and N-ethylmorpholinium salt.

In certain embodiments, Desidustat or pharmaceutically acceptable salts thereof, in a concentration selected from 1 mg/mL to 250 mg/mL. In certain embodiments, Desidustat or pharmaceutically acceptable salts thereof, in a concentration selected from 1 mg/mL to 200 mg/mL. In certain embodiments, Desidustat or pharmaceutically acceptable salts thereof, in a concentration selected from 1 mg/mL to 150 mg/mL. In certain embodiments, Desidustat or pharmaceutically acceptable salts thereof, in a concentration selected from 1 mg/mL to 125 mg/mL. In certain embodiments, Desidustat or pharmaceutically acceptable salts thereof, in a concentration selected from 1 mg/mL to 100 mg/mL. In certain embodiments, Desidustat or pharmaceutically acceptable salts thereof, in a concentration selected from 1 mg/mL to 75 mg/mL. In certain embodiments, Desidustat or pharmaceutically acceptable salts thereof, in a concentration selected from 1 mg/mL to 50 mg/mL. In certain embodiments, Desidustat or pharmaceutically acceptable salts thereof, in a concentration selected from 1 mg/mL to 25 mg/mL. In certain embodiments, Desidustat or pharmaceutically acceptable salts thereof, in a concentration selected from 1 mg/mL to 10 mg/mL. In certain embodiments, Desidustat or pharmaceutically acceptable salts thereof in a concentration selected from 25 mg/mL, 40 mg/mL, 50 mg/mL and 100 mg/mL.

Further, concentration of Desidustat or pharmaceutically acceptable salts thereof in parenteral pharmaceutical composition is selected from 1 mg/mL to 10 mg/mL, 10 mg/mL to 20 mg/mL, 20 mg/mL to 30 mg/mL, 30 mg/mL to 40 mg/mL, 40 mg/mL to 50 mg/mL, 50 mg/mL to 60 mg/mL, 60 mg/mL to 70 mg/mL, 70 mg/mL to 80 mg/mL, 80 mg/mL to 90 mg/mL, 90 mg/mL to 100 mg/mL, 100 mg/mL to 110 mg/mL, 110 mg/mL to 120 mg/mL, 120 mg/mL to 130 mg/mL, 130 mg/mL to 140 mg/mL, 140 mg/mL to 150 mg/mL, 150 mg/mL to 160 mg/mL, 160 mg/mL to 170 mg/mL, 170 mg/mL to 180 mg/mL, 180 mg/mL to 190 mg/mL, 190 mg/mL to 200 mg/mL, 200 mg/mL to 210 mg/mL, 210 mg/mL to 220 mg/mL, 220 mg/mL to 230 mg/mL, 230 mg/mL to 240 mg/mL and 240 mg/mL to 250 mg/mL.

Preferably, concentration of Desidustat or pharmaceutically acceptable salts thereof in parenteral pharmaceutical composition is selected from 1 mg/mL to 2 mg/mL, 2 mg/mL to 3 mg/mL, 3 mg/mL to 4 mg/mL, 4 mg/mL to 5 mg/mL, 5 mg/mL to 6 mg/mL, 6 mg/mL to 7 mg/mL, 7 mg/mL to 8 mg/mL, 8 mg/mL to 9 mg/mL, 9 mg/mL to 10 mg/mL, 10 mg/mL to 11 mg/mL, 11 mg/mL to 12 mg/mL, 12 mg/mL to 13 mg/mL, 13 mg/mL to 14 mg/mL, 14 mg/mL to 15 mg/mL, 15 mg/mL to 16 mg/mL, 16 mg/mL to 17 mg/mL, 17 mg/mL to 18 mg/mL, 18 mg/mL to 19 mg/mL, 19 mg/mL to 20 mg/mL, 20 mg/mL to 21 mg/mL, 21 mg/mL to 22 mg/mL, 22 mg/mL to 23 mg/mL, 23 mg/mL to 24 mg/mL, 24 mg/mL to 25 mg/mL, 25 mg/mL to 26 mg/mL, 26 mg/mL to 27 mg/mL, 27 mg/mL to 28 mg/mL, 28 mg/mL to 29 mg/mL, 29 mg/mL to 30 mg/mL, 30 mg/mL to 31 mg/mL, 31 mg/mL to 32 mg/mL, 32 mg/mL to 33 mg/mL, 33 mg/mL to 34 mg/mL, 34 mg/mL to 35 mg/mL, 35 mg/mL to 36 mg/mL, 36 mg/mL to 37 mg/mL, 37 mg/mL to 38 mg/mL, 38 mg/mL to 39 mg/mL, 39 mg/mL to 40 mg/mL, 40 mg/mL to 41 mg/mL, 41 mg/mL to 42 mg/mL, 42 mg/mL to 43 mg/mL, 43 mg/mL to 44 mg/mL, 44 mg/mL to 45 mg/mL, 45 mg/mL to 46 mg/mL, 46 mg/mL to 47 mg/mL, 47 mg/mL to 48 mg/mL, 48 mg/mL to 49 mg/mL, 49 mg/mL to 50 mg/mL, 50 mg/mL to 51 mg/mL, 51 mg/mL to 52 mg/mL, 52 mg/mL to 53 mg/mL, 53 mg/mL to 54 mg/mL, 54 mg/mL to 55 mg/mL, 55 mg/mL to 56 mg/mL, 56 mg/mL to 57 mg/mL, 57 mg/mL to 58 mg/mL, 58 mg/mL to 59 mg/mL, 59 mg/mL to 60 mg/mL, 60 mg/mL to 61 mg/mL, 61 mg/mL to 62 mg/mL, 62 mg/mL to 63 mg/mL, 63 mg/mL to 64 mg/mL, 64 mg/mL to 65 mg/mL, 65 mg/mL to 66 mg/mL, 66 mg/mL to 67 mg/mL, 67 mg/mL to 68 mg/mL, 68 mg/mL to 69 mg/mL, 69 mg/mL to 70 mg/mL, 70 mg/mL to 71 mg/mL, 71 mg/mL to 72 mg/mL, 72 mg/mL to 73 mg/mL, 73 mg/mL to 74 mg/mL, 74 mg/mL to 75 mg/mL, 75 mg/mL to 76 mg/mL, 76 mg/mL to 77 mg/mL, 77 mg/mL to 78 mg/mL, 78 mg/mL to 79 mg/mL, 79 mg/mL to 80 mg/mL, 80 mg/mL to 81 mg/mL, 81 mg/mL to 82 mg/mL, 82 mg/mL to 83 mg/mL, 83 mg/mL to 84 mg/mL, 84 mg/mL to 85 mg/mL, 85 mg/mL to 86 mg/mL, 86 mg/mL to 87 mg/mL, 87 mg/mL to 88 mg/mL, 88 mg/mL to 89 mg/mL, 89 mg/mL to 90 mg/mL, 90 mg/mL to 91 mg/mL, 91 mg/mL to 92 mg/mL, 92 mg/mL to 93 mg/mL, 93 mg/mL to 94 mg/mL, 94 mg/mL to 95 mg/mL, 95 mg/mL to 96 mg/mL, 96 mg/mL to 97 mg/mL, 97 mg/mL to 98 mg/mL, 98 mg/mL to 99 mg/mL, 99 mg/mL to 100 mg/mL, 100 mg/mL to 101 mg/mL, 101 mg/mL to 102 mg/mL, 102 mg/mL to 103 mg/mL, 103 mg/mL to 104 mg/mL, 104 mg/mL to 105 mg/mL, 105 mg/mL to 106 mg/mL, 106 mg/mL to 107 mg/mL, 107 mg/mL to 108 mg/mL, 108 mg/mL to 109 mg/mL, 109 mg/mL to 110 mg/mL, 110 mg/mL to 111 mg/mL, 111 mg/mL to 112 mg/mL, 112 mg/mL to 113 mg/mL, 113 mg/mL to 114 mg/mL, 114 mg/mL to 115 mg/mL, 115 mg/mL to 116 mg/mL, 116 mg/mL to 117 mg/mL, 117 mg/mL to 118 mg/mL, 118 mg/mL to 119 mg/mL, 119 mg/mL to 120 mg/mL, 120 mg/mL to 121 mg/mL, 121 mg/mL to 122 mg/mL, 122 mg/mL to 123 mg/mL, 123 mg/mL to 124 mg/mL, 124 mg/mL to 125 mg/mL, 125 mg/mL to 126 mg/mL, 126 mg/mL to 127 mg/mL, 127 mg/mL to 128 mg/mL, 128 mg/mL to 129 mg/mL, 129 mg/mL to 130 mg/mL, 130 mg/mL to 131 mg/mL, 131 mg/mL to 132 mg/mL, 132 mg/mL to 133 mg/mL, 133 mg/mL to 134 mg/mL, 134 mg/mL to 135 mg/mL, 135 mg/mL to 136 mg/mL, 136 mg/mL to 137 mg/mL, 137 mg/mL to 138 mg/mL, 138 mg/mL to 139 mg/mL, 139 mg/mL to 140 mg/mL, 140 mg/mL to 141 mg/mL, 141 mg/mL to 142 mg/mL, 142 mg/mL to 143 mg/mL, 143 mg/mL to 144 mg/mL, 144 mg/mL to 145 mg/mL, 145 mg/mL to 146 mg/mL, 146 mg/mL to 147 mg/mL, 147 mg/mL to 148 mg/mL, 148 mg/mL to 149 mg/mL and 149 mg/mL to 150 mg/mL.

More preferably, concentration of Desidustat or pharmaceutically acceptable salts thereof in parenteral pharmaceutical composition is selected from 1 mg/mL to 1.5 mg/mL, 1.5 mg/mL to 2 mg/mL, 2 mg/mL to 2.5 mg/mL, 2.5 mg/mL to 3 mg/mL, 3 mg/mL to 3.5 mg/mL, 3.5 mg/mL to 4 mg/mL, 4 mg/mL to 4.5 mg/mL, 4.5 mg/mL to 5 mg/mL, 5 mg/mL to 5.5 mg/mL, 5.5 mg/mL to 6 mg/mL, 6 mg/mL to 6.5 mg/mL, 6.5 mg/mL to 7 mg/mL, 7 mg/mL to 7.5 mg/mL, 7.5 mg/mL to 8 mg/mL, 8 mg/mL to 8.5 mg/mL, 8.5 mg/mL to 9 mg/mL, 9 mg/mL to 9.5 mg/mL, 9.5 mg/mL to 10 mg/mL, 10 mg/mL to 10.5 mg/mL, 10.5 mg/mL to 11 mg/mL, 11 mg/mL to 11.5 mg/mL, 11.5 mg/mL to 12 mg/mL, 12 mg/mL to 12.5 mg/mL, 12.5 mg/mL to 13 mg/mL, 13 mg/mL to 13.5 mg/mL, 13.5 mg/mL to 14 mg/mL, 14 mg/mL to 14.5 mg/mL, 14.5 mg/mL to 15 mg/mL, 15 mg/mL to 15.5 mg/mL, 15.5 mg/mL to 16 mg/mL, 16 mg/mL to 16.5 mg/mL, 16.5 mg/mL to 17 mg/mL, 17 mg/mL to 17.5 mg/mL, 17.5 mg/mL to 18 mg/mL, 18 mg/mL to 18.5 mg/mL, 18.5 mg/mL to 19 mg/mL, 19 mg/mL to 19.5 mg/mL, 19.5 mg/mL to 20 mg/mL, 20 mg/mL to 20.5 mg/mL, 20.5 mg/mL to 21 mg/mL, 21 mg/mL to 21.5 mg/mL, 21.5 mg/mL to 22 mg/mL, 22 mg/mL to 22.5 mg/mL, 22.5 mg/mL to 23 mg/mL, 23 mg/mL to 23.5 mg/mL, 23.5 mg/mL to 24 mg/mL, 24 mg/mL to 24.5 mg/mL, 24.5 mg/mL to 25 mg/mL, 25 mg/mL to 25.5 mg/mL, 25.5 mg/mL to 26 mg/mL, 26 mg/mL to 26.5 mg/mL, 26.5 mg/mL to 27 mg/mL, 27 mg/mL to 27.5 mg/mL, 27.5 mg/mL to 28 mg/mL, 28 mg/mL to 28.5 mg/mL, 28.5 mg/mL to 29 mg/mL, 29 mg/mL to 29.5 mg/mL, 29.5 mg/mL to 30 mg/mL, 30 mg/mL to 30.5 mg/mL, 30.5 mg/mL to 31 mg/mL, 31 mg/mL to 31.5 mg/mL, 31.5 mg/mL to 32 mg/mL, 32 mg/mL to 32.5 mg/mL, 32.5 mg/mL to 33 mg/mL, 33 mg/mL to 33.5 mg/mL, 33.5 mg/mL to 34 mg/mL, 34 mg/mL to 34.5 mg/mL, 34.5 mg/mL to 35 mg/mL, 35 mg/mL to 35.5 mg/mL, 35.5 mg/mL to 36 mg/mL, 36 mg/mL to 36.5 mg/mL, 36.5 mg/mL to 37 mg/mL, 37 mg/mL to 37.5 mg/mL, 37.5 mg/mL to 38 mg/mL, 38 mg/mL to 38.5 mg/mL, 38.5 mg/mL to 39 mg/mL, 39 mg/mL to 39.5 mg/mL, 39.5 mg/mL to 40 mg/mL, 40 mg/mL to 40.5 mg/mL, 40.5 mg/mL to 41 mg/mL, 41 mg/mL to 41.5 mg/mL, 41.5 mg/mL to 42 mg/mL, 42 mg/mL to 42.5 mg/mL, 42.5 mg/mL to 43 mg/mL, 43 mg/mL to 43.5 mg/mL, 43.5 mg/mL to 44 mg/mL, 44 mg/mL to 44.5 mg/mL, 44.5 mg/mL to 45 mg/mL, 45 mg/mL to 45.5 mg/mL, 45.5 mg/mL to 46 mg/mL, 46 mg/mL to 46.5 mg/mL, 46.5 mg/mL to 47 mg/mL, 47 mg/mL to 47.5 mg/mL, 47.5 mg/mL to 48 mg/mL, 48 mg/mL to 48.5 mg/mL, 48.5 mg/mL to 49 mg/mL, 49 mg/mL to 49.5 mg/mL and 49.5 mg/mL to 50 mg/mL.

In some embodiments, concentration of Desidustat or pharmaceutically acceptable salts thereof in parenteral pharmaceutical composition is in range of 15 mg/mL to 35 mg/mL. In some embodiments, concentration of Desidustat or pharmaceutically acceptable salts thereof in parenteral pharmaceutical composition is in range of 30 mg/mL to 50 mg/mL. In some embodiments, concentration of Desidustat or pharmaceutically acceptable salts thereof in parenteral pharmaceutical composition is in range of 40 mg/mL to 60 mg/mL. In some embodiments, concentration of Desidustat or pharmaceutically acceptable salts thereof in parenteral pharmaceutical composition is in range of 90 mg/mL to 110 mg/mL.

In some embodiments, concentration of Desidustat or pharmaceutically acceptable salts thereof in parenteral pharmaceutical composition is 25 mg/mL. In some embodiments, concentration of Desidustat or pharmaceutically acceptable salts thereof in parenteral pharmaceutical composition is 40 mg/mL. In some embodiments, concentration of Desidustat or pharmaceutically acceptable salts thereof in parenteral pharmaceutical composition is 50 mg/mL. In some embodiments, concentration of Desidustat or pharmaceutically acceptable salts thereof in parenteral pharmaceutical composition is 100 mg/mL.

In some embodiments, a parenteral pharmaceutical composition as described in any one of Examples 1-8. In some embodiments, a parenteral pharmaceutical composition as described in Example 1. In some embodiments, a parenteral pharmaceutical composition as described in Example 2. In some embodiments, a parenteral pharmaceutical composition as described in Example 3. In some embodiments, a parenteral pharmaceutical composition as described in Example 4. In some embodiments, a parenteral pharmaceutical composition as described in Example 5. In some embodiments, a parenteral pharmaceutical composition as described in Example 6. In some embodiments, a parenteral pharmaceutical composition as described in Example 7. In some embodiments, a parenteral pharmaceutical composition as described in Example 8.

Suitable pharmaceutically acceptable excipients selected from but are not limited to an isotonic agent/cosolvent and an alkalizing agent.

An isotonic agent/cosolvent selected from but are not limited to Albumin, Dimethylacetamide, Glycerin, Propylene Glycol, Polyethylene Glycol, PEG 200, PEG 300, PEG 400, Sodium Chloride, Mannitol, Trehalose, Dextrose, Hydroxypropyl Betadex, Potassium Chloride, Sulfobutylether O-Cyclodextrin, and Butylene Glycol or suitable mixture thereof.

In an aspect, the present invention relates to a parenteral composition of Desidustat or pharmaceutically acceptable salts thereof wherein an amount of isotonic agent/cosolvent used is in the range of 0.001% to 20% w/v.

Further, amount of isotonic agent/cosolvent in pharmaceutical composition used is in the range of 0.001% to 0.005%, 0.005% to 0.01%, 0.01% to 0.02%, 0.02% to 0.03%, 0.03% to 0.04%, 0.04% to 0.05%, 0.05% to 0.06%, 0.06% to 0.07%, 0.07% to 0.08%, 0.08% to 0.09%, 0.09% to 0.1%, 0.1% to 0.5%, 0.5% to 1%, 1% to 1.5%, 1.5% to 2%, 2% to 2.5%, 2.5% to 3%, 3% to 3.5%, 3.5% to 4%, 4% to 4.5%, 4.5% to 5%, 5% to 5.5%, 5.5% to 6%, 6% to 6.5%, 6.5% to 7%, 7% to 7.5%, 7.5% to 8%, 8% to 8.5%, 8.5% to 9%, 9% to 9.5%, 9.5% to 10%, 10% to 10.5%, 10.5% to 11%, 11% to 11.5%, 11.5% to 12%, 12% to 12.5%, 12.5% to 13%, 13% to 13.5%, 13.5% to 14%, 14% to 14.5%, 14.5% to 15%, 15% to 15.5%, 15.5% to 16%, 16% to 16.5%, 16.5% to 17%, 17% to 17.5%, 17.5% to 18%, 18% to 18.5%, 18.5% to 19%, 19% to 19.5% and 19.5% to 20%.

In certain embodiments, the amount of isotonic agent/cosolvent in pharmaceutical composition used is in the range of 0.01% to 0.05%, 0.05% to 0.1%, 0.1% to 0.15%, 0.15% to 0.2%, 0.2% to 0.25%, 0.25% to 0.3%, 0.3% to 0.35%, 0.35% to 0.4%, 0.4% to 0.45%, 0.45% to 0.5%, 0.5% to 0.55%, 0.55% to 0.6%, 0.6% to 0.65%, 0.65% to 0.7%, 0.7% to 0.75%, 0.75% to 0.8%, 0.8% to 0.85%, 0.85% to 0.9%, 0.9% to 0.95%, 0.95% to 1%, 1% to 1.05%, 1.05% to 1.1%, 1.1% to 1.15%, 1.15% to 1.2%, 1.2% to 1.25%, 1.25% to 1.3%, 1.3% to 1.35%, 1.35% to 1.4%, 1.4% to 1.45%, 1.45% to 1.5%, 1.5% to 1.55%, 1.55% to 1.6%, 1.6% to 1.65%, 1.65% to 1.7%, 1.7% to 1.75%, 1.75% to 1.8%, 1.8% to 1.85%, 1.85% to 1.9%, 1.9% to 1.95%, 1.95% to 2%, 2% to 2.15%, 2.15% to 2.2%, 2.2% to 2.25%, 2.25% to 2.3%, 2.3% to 2.35%, 2.35% to 2.4%, 2.4% to 2.45%, 2.45% to 2.5%, 2.5% to 2.55%, 2.55% to 2.6%, 2.6% to 2.65%, 2.65% to 2.7%, 2.7% to 2.75%, 2.75% to 2.8%, 2.8% to 2.85%, 2.85% to 2.9%, 2.9% to 2.95%, 2.95% to 3%, 3% to 3.05%, 3.05% to 3.1%, 3.1% to 3.15%, 3.15% to 3.2%, 3.2% to 3.25%, 3.25% to 3.3%, 3.3% to 3.35%, 3.35% to 3.4%, 3.4% to 3.45%, 3.45% to 3.5%, 3.5% to 3.55%, 3.55% to 3.6%, 3.6% to 3.65%, 3.65% to 3.7%, 3.7% to 3.75%, 3.75% to 3.8%, 3.8% to 3.85%, 3.85% to 3.9%, 3.9% to 3.95%, 3.95% to 4%, 4% to 4.05%, 4.05% to 4.1%, 4.1% to 4.15%, 4.15% to 4.2%, 4.2% to 4.25%, 4.25% to 4.3%, 4.3% to 4.35%, 4.35% to 4.4%, 4.4% to 4.45%, 4.45% to 4.5%, 4.5% to 4.55%, 4.55% to 4.6%, 4.6% to 4.65%, 4.65% to 4.7%, 4.7% to 4.75%, 4.75% to 4.8%, 4.8% to 4.85%, 4.85% to 4.9%, 4.9% to 4.95% and 4.95% to 5%.

In certain embodiments, the amount of isotonic agent/cosolvent in pharmaceutical composition used is in the range of 0.1% to 1.5%.

An alkalizing agent may be selected from but is not limited to Triethanolamine (Trolamine), Calcium Hydroxide, Diethanolamine, Ethanolamine (Monoethanolamine), Potassium Hydroxide, Sodium Carbonate, Sodium Citrate, Sodium Hydroxide, Meglumine, Sodium lactate, Tromethamine or suitable mixture thereof.

In an aspect, the present invention relates to a parenteral composition of Desidustat or pharmaceutically acceptable salts thereof wherein an amount of alkalizing agent used is in the range of 0.001% to 20% w/v.

Further, amount of alkalizing agent in pharmaceutical composition used is in the range of 0.001% to 0.005%, 0.005% to 0.01%, 0.01% to 0.02%, 0.02% to 0.03%, 0.03% to 0.04%, 0.04% to 0.05%, 0.05% to 0.06%, 0.06% to 0.07%, 0.07% to 0.08%, 0.08% to 0.09%, 0.09% to 0.1%, 0.1% to 0.5%, 0.5% to 1%, 1% to 1.5%, 1.5% to 2%, 2% to 2.5%, 2.5% to 3%, 3% to 3.5%, 3.5% to 4%, 4% to 4.5%, 4.5% to 5%, 5% to 5.5%, 5.5% to 6%, 6% to 6.5%, 6.5% to 7%, 7% to 7.5%, 7.5% to 8%, 8% to 8.5%, 8.5% to 9%, 9% to 9.5%, 9.5% to 10%, 10% to 10.5%, 10.5% to 11%, 11% to 11.5%, 11.5% to 12%, 12% to 12.5%, 12.5% to 13%, 13% to 13.5%, 13.5% to 14%, 14% to 14.5%, 14.5% to 15%, 15% to 15.5%, 15.5% to 16%, 16% to 16.5%, 16.5% to 17%, 17% to 17.5%, 17.5% to 18%, 18% to 18.5%, 18.5% to 19%, 19% to 19.5% and 19.5% to 20%.

In certain embodiments, the amount of alkalizing agent used in the pharmaceutical composition is in the range of 0.01% to 0.05%, 0.05% to 0.1%, 0.1% to 0.15%, 0.15% to 0.2%, 0.2% to 0.25%, 0.25% to 0.3%, 0.3% to 0.35%, 0.35% to 0.4%, 0.4% to 0.45%, 0.45% to 0.5%, 0.5% to 0.55%, 0.55% to 0.6%, 0.6% to 0.65%, 0.65% to 0.7%, 0.7% to 0.75%, 0.75% to 0.8%, 0.8% to 0.85%, 0.85% to 0.9%, 0.9% to 0.95%, 0.95% to 1%, 1% to 1.05%, 1.05% to 1.1%, 1.1% to 1.15%, 1.15% to 1.2%, 1.2% to 1.25%, 1.25% to 1.3%, 1.3% to 1.35%, 1.35% to 1.4%, 1.4% to 1.45%, 1.45% to 1.5%, 1.5% to 1.55%, 1.55% to 1.6%, 1.6% to 1.65%, 1.65% to 1.7%, 1.7% to 1.75%, 1.75% to 1.8%, 1.8% to 1.85%, 1.85% to 1.9%, 1.9% to 1.95%, 1.95% to 2%, 2% to 2.15%, 2.15% to 2.2%, 2.2% to 2.25%, 2.25% to 2.3%, 2.3% to 2.35%, 2.35% to 2.4%, 2.4% to 2.45%, 2.45% to 2.5%, 2.5% to 2.55%, 2.55% to 2.6%, 2.6% to 2.65%, 2.65% to 2.7%, 2.7% to 2.75%, 2.75% to 2.8%, 2.8% to 2.85%, 2.85% to 2.9%, 2.9% to 2.95%, 2.95% to 3%, 3% to 3.05%, 3.05% to 3.1%, 3.1% to 3.15%, 3.15% to 3.2%, 3.2% to 3.25%, 3.25% to 3.3%, 3.3% to 3.35%, 3.35% to 3.4%, 3.4% to 3.45%, 3.45% to 3.5%, 3.5% to 3.55%, 3.55% to 3.6%, 3.6% to 3.65%, 3.65% to 3.7%, 3.7% to 3.75%, 3.75% to 3.8%, 3.8% to 3.85%, 3.85% to 3.9%, 3.9% to 3.95%, 3.95% to 4%, 4% to 4.05%, 4.05% to 4.1%, 4.1% to 4.15%, 4.15% to 4.2%, 4.2% to 4.25%, 4.25% to 4.3%, 4.3% to 4.35%, 4.35% to 4.4%, 4.4% to 4.45%, 4.45% to 4.5%, 4.5% to 4.55%, 4.55% to 4.6%, 4.6% to 4.65%, 4.65% to 4.7%, 4.7% to 4.75%, 4.75% to 4.8%, 4.8% to 4.85%, 4.85% to 4.9%, 4.9% to 4.95% and 4.95% to 5%.

In certain embodiments, the amount of alkalizing agent in pharmaceutical composition used is in the range of 0.5% to 3%.

Final parenteral pharmaceutical composition of Desidustat can be made by adding water.

### General Process for the Preparation of Parenteral Composition of Desidustat

### Preparation of Parenteral Composition of Desidustat

1. Accurate quantity of Water for Injection (WFI) was taken in a suitable container.
2. A weighted quantity of suitable alkalizing agent was added into the container under stirring, and stirring was continued until it formed a clear solution.
3. A weighted quantity of suitable isotonic agent was added into the above container under stirring, and stirring was continued until it formed a clear solution.
4. Gradually, added weighed quantity of Desidustat into the above container under stirring, and stirring continued until Desidustat gets solubilized.
5. The volume of the above solution was made up to batch size using WFI and mixed well.
6. The above solution was filtered through a 0.2 μm filter.
7. The solution was filled into clear USP type 1 glass vials, plugged with 20 mm rubber stopper and sealed with 20 mm aluminium flip off seal.

The invention is further exemplified by the non-limiting examples described herein, which are illustrative representing the preferred modes of carrying out the invention. The invention's scope is not limited to these specific embodiments only but should be read in conjunction with what is disclosed anywhere else in the specification together with that information and knowledge, which are within the general understanding of a person skilled in the art. The parenteral pharmaceutical composition of Desidustat is exemplified in examples 1 to 8.

### Method of Treating Radiation Induced Toxicity by Desidustat or Pharmaceutically Acceptable Salts Thereof

In an embodiment the present invention provides a method of treating radiation induced toxicity in a subject, comprises administering an effective dose of Desidustat or a pharmaceutically acceptable salt thereof.

In an embodiment, radiation induced toxicity includes acute radiation syndrome.

In another embodiment, Acute Radiation Syndrome is selected from Hematopoietic Acute Radiation Syndrome (H-ARS).

Hematopoietic Acute Radiation Syndrome occurs when the whole body or a significant portion of body is exposed to a high dose of penetrating ionizing radiation, typically between 0.7 and 10 Gray (Gy), delivered over a short period (minutes to hours).

Desidustat is useful for the treatment of Hematopoietic Acute Radiation Syndrome.

The suitable pharmaceutically acceptable salts of Desidustat may be selected from suitable inorganic metal salt or an organic amine salt.

In certain embodiments, the inorganic metal salts can be selected from calcium salt, sodium salt, potassium salt, lithium salt, barium salt, strontium salt, magnesium salt, cesium salt, copper salt, cobalt salt, iron salt, manganese salt, lead salt, aluminium salt, cadmium salt, silver salt, and zinc salt, and the like.

In certain embodiments, the organic amine salts, also referred to herein as organic ammonium salts can be selected from methylammonium salt, dimethylammonium salt, ethylammonium salt, diethyl ammonium salt, n-propyl ammonium salt, isopropyl ammonium salt, diisopropyl ammonium salt, N-methyl isopropyl ammonium salt, n-butyl ammonium salt, t-butyl ammonium salt, 2-butammonium salt, 1,2-ethane diammonium salt, N-methylglucammonium salt, N,N,N-trimethyl ethanolammonium hydroxide (choline) salt, tromethammmonium salt, cyclohexylammonium salt, N-methyl cyclohexylammonium salt, guanidinium salt, N-(4-aminobutyl) guanidinium salt, dicyclohexylammonium salt, benzene-methanammonium salt, ethanolammonium salt, diethanolammonium salt, tris-(hydroxymethyl)methylammonium salt, hydroxylammonium salt, methanammonium salt, benzylammonium salt, N-methylbenzylammonium salt, N-ethyl benzylammonium salt, 4-methoxybenzylammonium salt, pyrrolidinium salt, piperidinium salt, piperazinium salt, morpholinium salt, 2-aminopyrimidinium salt, alaninium salt, lysinium salt, argininium salt, histidinium salt, threoninium salt, prolinium salt, glutammonium salt, glycinium salt, 2-thiopheneethanammonium salt, (2S)-3,3-dimethyl-2-butanammonium salt, cyclopentanammonium salt, cycloheptanammonium salt, meglummonium salt, bethammonium salt, dibenzylammonium salt, diphenylammonium salt, α-naphthylammonium salt, O-phenylenediammonium salt, 1,3-Diaminopropammonium salt, (S)-α-naphthylethylammonium salt, (S)-3-methoxyphenylethylammonium salt, (S)-4-methoxyphenylethylammonium salt, (S)-4-chlorophenylethylammonium salt, (S)-4-methylphenylethylammonium salt, cinchoninium salt, cinchonidinium salt, (−)-quininium salt, triethanolammonium salt, imidazolium salt, ethylenediammonium salt, polammonium salt, morpholinium 4-(2-hydroxyethyl) salt, N—N-diethylethanolammonium salt, deammonium salt, hydrabammonium salt, betammonium salt, adamantanammonium salt, L-adamantanmethylammonium salt, tritylammonium salt, glucammonium salt, N-methyl pyrrolidinium salt, ureammonium salt, procammonium salt, metformmonium salt, hexane1-6-diammonium salt, 2-(2-aminoethoxy)ethanammonium salt, N-methylmorpholinium salt, and N-ethylmorpholinium salt.

In certain embodiments, effective amount of Desidustat or pharmaceutically acceptable salts thereof, selected from 1 mg to 500 mg. In certain embodiments, effective amount of Desidustat or pharmaceutically acceptable salts thereof, selected from 1 mg to 400 mg. In certain embodiments, effective amount of Desidustat or pharmaceutically acceptable salts thereof, selected from 1 mg to 300 mg. In certain embodiments, effective amount of Desidustat or pharmaceutically acceptable salts thereof, selected from 1 mg to 250 mg. In certain embodiments, effective amount of Desidustat or pharmaceutically acceptable salts thereof, selected from 1 mg to 200 mg. In certain embodiments, effective amount of Desidustat or pharmaceutically acceptable salts thereof, selected from 1 mg to 150 mg. In certain embodiments, effective amount of Desidustat or pharmaceutically acceptable salts thereof, selected from 1 mg to 100 mg. In certain embodiments, effective amount of Desidustat or pharmaceutically acceptable salts thereof, selected from 1 mg to 75 mg. In certain embodiments, Desidustat or pharmaceutically acceptable salts thereof, in a concentration selected from 1 mg to 50 mg In certain embodiments, effective amount of Desidustat or pharmaceutically acceptable salts thereof, selected from 1 mg to 25 mg. In certain embodiments, effective amount of Desidustat or pharmaceutically acceptable salts thereof, selected from 25 mg, 50 mg and 100 mg.

In certain embodiments, effective amount of Desidustat or pharmaceutically acceptable salts thereof, selected from 1 mg to 10 mg, 10 mg to 20 mg, 20 mg to 30 mg, 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 110 mg, 110 mg to 120 mg, 120 mg to 130 mg, 130 mg to 140 mg, 140 mg to 150 mg, 150 mg to 160 mg, 160 mg to 170 mg, 170 mg to 180 mg, 180 mg to 190 mg, 190 mg to 200 mg, 200 mg to 210 mg, 210 mg to 220 mg, 220 mg to 230 mg, 230 mg to 240 mg and 240 mg to 250 mg.

In certain embodiments, effective amount of Desidustat or pharmaceutically acceptable salts thereof, selected from 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, and 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg and 150 mg.

In certain embodiments, Desidustat or pharmaceutically acceptable salts thereof can be further characterized according to the dose of compound administered to a subject, where subject is animal or human.

In another embodiments, Desidustat is administered to the subject once a day, twice a day or thrice a day or on alternate day for at least 2 weeks.

In a further embodiment, the present invention provides effective dose of Desidustat or its pharmaceutically acceptable salts thereof is administered to subject by oral pharmaceutical composition or parenteral pharmaceutical composition.

In a further embodiment, use of Desidustat or pharmaceutically acceptable salts thereof in the manufacture of a medicament for the treatment of radiation induced toxicity.

### Method of Treating Radiation Induced Toxicity by Parenteral Pharmaceutical Composition of Desidustat or Pharmaceutically Acceptable Salts Thereof

In an embodiment, present invention provides a method of treating radiation induced toxicity in a subject by parenteral pharmaceutical composition of Desidustat or pharmaceutically acceptable salts thereof optionally with one or more pharmaceutically acceptable excipients.

In an embodiment, radiation induced toxicity includes acute radiation syndrome.

In another embodiment, Acute Radiation Syndrome is selected from Hematopoietic Acute Radiation Syndrome (H-ARS).

Hematopoietic Acute Radiation Syndrome occurs when the whole body or a significant portion of body is exposed to a high dose of penetrating ionizing radiation, typically between 0.7 and 10 Gray (Gy), delivered over a short period (minutes to hours).

The parenteral pharmaceutical composition of Desidustat is useful for the treatment of Hematopoietic Acute Radiation Syndrome.

The suitable pharmaceutically acceptable salts of Desidustat may be selected from suitable inorganic metal salt or an organic amine salt.

In certain embodiments, the inorganic metal salts can be selected from calcium salt, sodium salt, potassium salt, lithium salt, barium salt, strontium salt, magnesium salt, cesium salt, copper salt, cobalt salt, iron salt, manganese salt, lead salt, aluminium salt, cadmium salt, silver salt, and zinc salt, and the like.

In certain embodiments, the organic amine salts, also referred to herein as organic ammonium salts can be selected from methylammonium salt, dimethylammonium salt, ethylammonium salt, diethyl ammonium salt, n-propyl ammonium salt, isopropyl ammonium salt, diisopropyl ammonium salt, N-methyl isopropyl ammonium salt, n-butyl ammonium salt, t-butyl ammonium salt, 2-butammonium salt, 1,2-ethane diammonium salt, N-methylglucammonium salt, N,N,N-trimethyl ethanolammonium hydroxide (choline) salt, tromethammmonium salt, cyclohexylammonium salt, N-methyl cyclohexylammonium salt, guanidinium salt, N-(4-aminobutyl) guanidinium salt, dicyclohexylammonium salt, benzene-methanammonium salt, ethanolammonium salt, diethanolammonium salt, tris-(hydroxymethyl)methylammonium salt, hydroxylammonium salt, methanammonium salt, benzylammonium salt, N-methylbenzylammonium salt, N-ethyl benzylammonium salt, 4-methoxybenzylammonium salt, pyrrolidinium salt, piperidinium salt, piperazinium salt, morpholinium salt, 2-aminopyrimidinium salt, alaninium salt, lysinium salt, argininium salt, histidinium salt, threoninium salt, prolinium salt, glutammonium salt, glycinium salt, 2-thiopheneethanammonium salt, (2S)-3,3-dimethyl-2-butanammonium salt, cyclopentanammonium salt, cycloheptanammonium salt, meglummonium salt, bethammonium salt, dibenzylammonium salt, diphenylammonium salt, α-naphthylammonium salt, O-phenylenediammonium salt, 1,3-Diaminopropammonium salt, (S)-α-naphthylethylammonium salt, (S)-3-methoxyphenylethylammonium salt, (S)-4-methoxyphenylethylammonium salt, (S)-4-chlorophenylethylammonium salt, (S)-4-methylphenylethylammonium salt, cinchoninium salt, cinchonidinium salt, (−)-quininium salt, triethanolammonium salt, imidazolium salt, ethylenediammonium salt, polammonium salt, morpholinium 4-(2-hydroxyethyl) salt, N—N-diethylethanolammonium salt, deammonium salt, hydrabammonium salt, betammonium salt, adamantanammonium salt, L-adamantanmethylammonium salt, tritylammonium salt, glucammonium salt, N-methyl pyrrolidinium salt, ureammonium salt, procammonium salt, metformmonium salt, hexane1-6-diammonium salt, 2-(2-aminoethoxy)ethanammonium salt, N-methylmorpholinium salt, and N-ethylmorpholinium salt.

In certain embodiments, Desidustat or pharmaceutically acceptable salts thereof, in a concentration selected from 1 mg/mL to 250 mg/mL. In certain embodiments, Desidustat or pharmaceutically acceptable salts thereof, in a concentration selected from 1 mg/mL to 200 mg/mL. In certain embodiments, Desidustat or pharmaceutically acceptable salts thereof, in a concentration selected from 1 mg/mL to 150 mg/mL. In certain embodiments, Desidustat or pharmaceutically acceptable salts thereof, in a concentration selected from 1 mg/mL to 125 mg/mL. In certain embodiments, Desidustat or pharmaceutically acceptable salts thereof, in a concentration selected from 1 mg/mL to 100 mg/mL. In certain embodiments, Desidustat or pharmaceutically acceptable salts thereof, in a concentration selected from 1 mg/mL to 75 mg/mL. In certain embodiments, Desidustat or pharmaceutically acceptable salts thereof, in a concentration selected from 1 mg/mL to 50 mg/mL. In certain embodiments, Desidustat or pharmaceutically acceptable salts thereof, in a concentration selected from 1 mg/mL to 25 mg/mL. In certain embodiments, Desidustat or pharmaceutically acceptable salts thereof, in a concentration selected from 1 mg/mL to 10 mg/mL. In certain embodiments, Desidustat or pharmaceutically acceptable salts thereof in a concentration selected from 25 mg/mL, 40 mg/mL, 50 mg/mL and 100 mg/mL.

Further, concentration of Desidustat or pharmaceutically acceptable salts thereof in parenteral pharmaceutical composition is selected from 1 mg/mL to 10 mg/mL, 10 mg/mL to 20 mg/mL, 20 mg/mL to 30 mg/mL, 30 mg/mL to 40 mg/mL, 40 mg/mL to 50 mg/mL, 50 mg/mL to 60 mg/mL, 60 mg/mL to 70 mg/mL, 70 mg/mL to 80 mg/mL, 80 mg/mL to 90 mg/mL, 90 mg/mL to 100 mg/mL, 100 mg/mL to 110 mg/mL, 110 mg/mL to 120 mg/mL, 120 mg/mL to 130 mg/mL, 130 mg/mL to 140 mg/mL, 140 mg/mL to 150 mg/mL, 150 mg/mL to 160 mg/mL, 160 mg/mL to 170 mg/mL, 170 mg/mL to 180 mg/mL, 180 mg/mL to 190 mg/mL, 190 mg/mL to 200 mg/mL, 200 mg/mL to 210 mg/mL, 210 mg/mL to 220 mg/mL, 220 mg/mL to 230 mg/mL, 230 mg/mL to 240 mg/mL and 240 mg/mL to 250 mg/mL.

Preferably, concentration of Desidustat or pharmaceutically acceptable salts thereof in parenteral pharmaceutical composition is selected from 1 mg/mL to 2 mg/mL, 2 mg/mL to 3 mg/mL, 3 mg/mL to 4 mg/mL, 4 mg/mL to 5 mg/mL, 5 mg/mL to 6 mg/mL, 6 mg/mL to 7 mg/mL, 7 mg/mL to 8 mg/mL, 8 mg/mL to 9 mg/mL, 9 mg/mL to 10 mg/mL, 10 mg/mL to 11 mg/mL, 11 mg/mL to 12 mg/mL, 12 mg/mL to 13 mg/mL, 13 mg/mL to 14 mg/mL, 14 mg/mL to 15 mg/mL, 15 mg/mL to 16 mg/mL, 16 mg/mL to 17 mg/mL, 17 mg/mL to 18 mg/mL, 18 mg/mL to 19 mg/mL, 19 mg/mL to 20 mg/mL, 20 mg/mL to 21 mg/mL, 21 mg/mL to 22 mg/mL, 22 mg/mL to 23 mg/mL, 23 mg/mL to 24 mg/mL, 24 mg/mL to 25 mg/mL, 25 mg/mL to 26 mg/mL, 26 mg/mL to 27 mg/mL, 27 mg/mL to 28 mg/mL, 28 mg/mL to 29 mg/mL, 29 mg/mL to 30 mg/mL, 30 mg/mL to 31 mg/mL, 31 mg/mL to 32 mg/mL, 32 mg/mL to 33 mg/mL, 33 mg/mL to 34 mg/mL, 34 mg/mL to 35 mg/mL, 35 mg/mL to 36 mg/mL, 36 mg/mL to 37 mg/mL, 37 mg/mL to 38 mg/mL, 38 mg/mL to 39 mg/mL, 39 mg/mL to 40 mg/mL, 40 mg/mL to 41 mg/mL, 41 mg/mL to 42 mg/mL, 42 mg/mL to 43 mg/mL, 43 mg/mL to 44 mg/mL, 44 mg/mL to 45 mg/mL, 45 mg/mL to 46 mg/mL, 46 mg/mL to 47 mg/mL, 47 mg/mL to 48 mg/mL, 48 mg/mL to 49 mg/mL, 49 mg/mL to 50 mg/mL, 50 mg/mL to 51 mg/mL, 51 mg/mL to 52 mg/mL, 52 mg/mL to 53 mg/mL, 53 mg/mL to 54 mg/mL, 54 mg/mL to 55 mg/mL, 55 mg/mL to 56 mg/mL, 56 mg/mL to 57 mg/mL, 57 mg/mL to 58 mg/mL, 58 mg/mL to 59 mg/mL, 59 mg/mL to 60 mg/mL, 60 mg/mL to 61 mg/mL, 61 mg/mL to 62 mg/mL, 62 mg/mL to 63 mg/mL, 63 mg/mL to 64 mg/mL, 64 mg/mL to 65 mg/mL, 65 mg/mL to 66 mg/mL, 66 mg/mL to 67 mg/mL, 67 mg/mL to 68 mg/mL, 68 mg/mL to 69 mg/mL, 69 mg/mL to 70 mg/mL, 70 mg/mL to 71 mg/mL, 71 mg/mL to 72 mg/mL, 72 mg/mL to 73 mg/mL, 73 mg/mL to 74 mg/mL, 74 mg/mL to 75 mg/mL, 75 mg/mL to 76 mg/mL, 76 mg/mL to 77 mg/mL, 77 mg/mL to 78 mg/mL, 78 mg/mL to 79 mg/mL, 79 mg/mL to 80 mg/mL, 80 mg/mL to 81 mg/mL, 81 mg/mL to 82 mg/mL, 82 mg/mL to 83 mg/mL, 83 mg/mL to 84 mg/mL, 84 mg/mL to 85 mg/mL, 85 mg/mL to 86 mg/mL, 86 mg/mL to 87 mg/mL, 87 mg/mL to 88 mg/mL, 88 mg/mL to 89 mg/mL, 89 mg/mL to 90 mg/mL, 90 mg/mL to 91 mg/mL, 91 mg/mL to 92 mg/mL, 92 mg/mL to 93 mg/mL, 93 mg/mL to 94 mg/mL, 94 mg/mL to 95 mg/mL, 95 mg/mL to 96 mg/mL, 96 mg/mL to 97 mg/mL, 97 mg/mL to 98 mg/mL, 98 mg/mL to 99 mg/mL, 99 mg/mL to 100 mg/mL, 100 mg/mL to 101 mg/mL, 101 mg/mL to 102 mg/mL, 102 mg/mL to 103 mg/mL, 103 mg/mL to 104 mg/mL, 104 mg/mL to 105 mg/mL, 105 mg/mL to 106 mg/mL, 106 mg/mL to 107 mg/mL, 107 mg/mL to 108 mg/mL, 108 mg/mL to 109 mg/mL, 109 mg/mL to 110 mg/mL, 110 mg/mL to 111 mg/mL, 111 mg/mL to 112 mg/mL, 112 mg/mL to 113 mg/mL, 113 mg/mL to 114 mg/mL, 114 mg/mL to 115 mg/mL, 115 mg/mL to 116 mg/mL, 116 mg/mL to 117 mg/mL, 117 mg/mL to 118 mg/mL, 118 mg/mL to 119 mg/mL, 119 mg/mL to 120 mg/mL, 120 mg/mL to 121 mg/mL, 121 mg/mL to 122 mg/mL, 122 mg/mL to 123 mg/mL, 123 mg/mL to 124 mg/mL, 124 mg/mL to 125 mg/mL, 125 mg/mL to 126 mg/mL, 126 mg/mL to 127 mg/mL, 127 mg/mL to 128 mg/mL, 128 mg/mL to 129 mg/mL, 129 mg/mL to 130 mg/mL, 130 mg/mL to 131 mg/mL, 131 mg/mL to 132 mg/mL, 132 mg/mL to 133 mg/mL, 133 mg/mL to 134 mg/mL, 134 mg/mL to 135 mg/mL, 135 mg/mL to 136 mg/mL, 136 mg/mL to 137 mg/mL, 137 mg/mL to 138 mg/mL, 138 mg/mL to 139 mg/mL, 139 mg/mL to 140 mg/mL, 140 mg/mL to 141 mg/mL, 141 mg/mL to 142 mg/mL, 142 mg/mL to 143 mg/mL, 143 mg/mL to 144 mg/mL, 144 mg/mL to 145 mg/mL, 145 mg/mL to 146 mg/mL, 146 mg/mL to 147 mg/mL, 147 mg/mL to 148 mg/mL, 148 mg/mL to 149 mg/mL and 149 mg/mL to 150 mg/mL.

More preferably, concentration of Desidustat or pharmaceutically acceptable salts thereof in parenteral pharmaceutical composition is selected from 1 mg/mL to 1.5 mg/mL, 1.5 mg/mL to 2 mg/mL, 2 mg/mL to 2.5 mg/mL, 2.5 mg/mL to 3 mg/mL, 3 mg/mL to 3.5 mg/mL, 3.5 mg/mL to 4 mg/mL, 4 mg/mL to 4.5 mg/mL, 4.5 mg/mL to 5 mg/mL, 5 mg/mL to 5.5 mg/mL, 5.5 mg/mL to 6 mg/mL, 6 mg/mL to 6.5 mg/mL, 6.5 mg/mL to 7 mg/mL, 7 mg/mL to 7.5 mg/mL, 7.5 mg/mL to 8 mg/mL, 8 mg/mL to 8.5 mg/mL, 8.5 mg/mL to 9 mg/mL, 9 mg/mL to 9.5 mg/mL, 9.5 mg/mL to 10 mg/mL, 10 mg/mL to 10.5 mg/mL, 10.5 mg/mL to 11 mg/mL, 11 mg/mL to 11.5 mg/mL, 11.5 mg/mL to 12 mg/mL, 12 mg/mL to 12.5 mg/mL, 12.5 mg/mL to 13 mg/mL, 13 mg/mL to 13.5 mg/mL, 13.5 mg/mL to 14 mg/mL, 14 mg/mL to 14.5 mg/mL, 14.5 mg/mL to 15 mg/mL, 15 mg/mL to 15.5 mg/mL, 15.5 mg/mL to 16 mg/mL, 16 mg/mL to 16.5 mg/mL, 16.5 mg/mL to 17 mg/mL, 17 mg/mL to 17.5 mg/mL, 17.5 mg/mL to 18 mg/mL, 18 mg/mL to 18.5 mg/mL, 18.5 mg/mL to 19 mg/mL, 19 mg/mL to 19.5 mg/mL, 19.5 mg/mL to 20 mg/mL, 20 mg/mL to 20.5 mg/mL, 20.5 mg/mL to 21 mg/mL, 21 mg/mL to 21.5 mg/mL, 21.5 mg/mL to 22 mg/mL, 22 mg/mL to 22.5 mg/mL, 22.5 mg/mL to 23 mg/mL, 23 mg/mL to 23.5 mg/mL, 23.5 mg/mL to 24 mg/mL, 24 mg/mL to 24.5 mg/mL, 24.5 mg/mL to 25 mg/mL, 25 mg/mL to 25.5 mg/mL, 25.5 mg/mL to 26 mg/mL, 26 mg/mL to 26.5 mg/mL, 26.5 mg/mL to 27 mg/mL, 27 mg/mL to 27.5 mg/mL, 27.5 mg/mL to 28 mg/mL, 28 mg/mL to 28.5 mg/mL, 28.5 mg/mL to 29 mg/mL, 29 mg/mL to 29.5 mg/mL, 29.5 mg/mL to 30 mg/mL, 30 mg/mL to 30.5 mg/mL, 30.5 mg/mL to 31 mg/mL, 31 mg/mL to 31.5 mg/mL, 31.5 mg/mL to 32 mg/mL, 32 mg/mL to 32.5 mg/mL, 32.5 mg/mL to 33 mg/mL, 33 mg/mL to 33.5 mg/mL, 33.5 mg/mL to 34 mg/mL, 34 mg/mL to 34.5 mg/mL, 34.5 mg/mL to 35 mg/mL, 35 mg/mL to 35.5 mg/mL, 35.5 mg/mL to 36 mg/mL, 36 mg/mL to 36.5 mg/mL, 36.5 mg/mL to 37 mg/mL, 37 mg/mL to 37.5 mg/mL, 37.5 mg/mL to 38 mg/mL, 38 mg/mL to 38.5 mg/mL, 38.5 mg/mL to 39 mg/mL, 39 mg/mL to 39.5 mg/mL, 39.5 mg/mL to 40 mg/mL, 40 mg/mL to 40.5 mg/mL, 40.5 mg/mL to 41 mg/mL, 41 mg/mL to 41.5 mg/mL, 41.5 mg/mL to 42 mg/mL, 42 mg/mL to 42.5 mg/mL, 42.5 mg/mL to 43 mg/mL, 43 mg/mL to 43.5 mg/mL, 43.5 mg/mL to 44 mg/mL, 44 mg/mL to 44.5 mg/mL, 44.5 mg/mL to 45 mg/mL, 45 mg/mL to 45.5 mg/mL, 45.5 mg/mL to 46 mg/mL, 46 mg/mL to 46.5 mg/mL, 46.5 mg/mL to 47 mg/mL, 47 mg/mL to 47.5 mg/mL, 47.5 mg/mL to 48 mg/mL, 48 mg/mL to 48.5 mg/mL, 48.5 mg/mL to 49 mg/mL, 49 mg/mL to 49.5 mg/mL and 49.5 mg/mL to 50 mg/mL.

In some embodiments, concentration of Desidustat or pharmaceutically acceptable salts thereof in parenteral pharmaceutical composition is in range of 15 mg/mL to 35 mg/mL. In some embodiments, concentration of Desidustat or pharmaceutically acceptable salts thereof in parenteral pharmaceutical composition is in range of 30 mg/mL to 50 mg/mL. In some embodiments, concentration of Desidustat or pharmaceutically acceptable salts thereof in parenteral pharmaceutical composition is in range of 40 mg/mL to 60 mg/mL. In some embodiments, concentration of Desidustat or pharmaceutically acceptable salts thereof in parenteral pharmaceutical composition is in range of 90 mg/mL to 110 mg/mL.

In some embodiments, concentration of Desidustat or pharmaceutically acceptable salts thereof in parenteral pharmaceutical composition is 25 mg/mL. In some embodiments, concentration of Desidustat or pharmaceutically acceptable salts thereof in parenteral pharmaceutical composition is 40 mg/mL. In some embodiments, concentration of Desidustat or pharmaceutically acceptable salts thereof in parenteral pharmaceutical composition is 50 mg/mL. In some embodiments, concentration of Desidustat or pharmaceutically acceptable salts thereof in parenteral pharmaceutical composition is 100 mg/mL.

Suitable pharmaceutically acceptable excipients selected from but are not limited to an isotonic agent/cosolvent and an alkalizing agent.

An isotonic agent/cosolvent selected from but are not limited to Albumin, Dimethylacetamide, Glycerin, Propylene Glycol, Polyethylene Glycol, PEG 200, PEG 300, PEG 400, Sodium Chloride, Mannitol, Trehalose, Dextrose, Hydroxypropyl Betadex, Potassium Chloride, Sulfobutylether O-Cyclodextrin, and Butylene Glycol or suitable mixture thereof.

In an aspect, the present invention relates to a parenteral composition of Desidustat or pharmaceutically acceptable salts thereof wherein an amount of isotonic agent/cosolvent used is in the range of 0.001% to 20% w/v.

Further, amount of isotonic agent/cosolvent in pharmaceutical composition used is in the range of 0.001% to 0.005%, 0.005% to 0.01%, 0.01% to 0.02%, 0.02% to 0.03%, 0.03% to 0.04%, 0.04% to 0.05%, 0.05% to 0.06%, 0.06% to 0.07%, 0.07% to 0.08%, 0.08% to 0.09%, 0.09% to 0.1%, 0.1% to 0.5%, 0.5% to 1%, 1% to 1.5%, 1.5% to 2%, 2% to 2.5%, 2.5% to 3%, 3% to 3.5%, 3.5% to 4%, 4% to 4.5%, 4.5% to 5%, 5% to 5.5%, 5.5% to 6%, 6% to 6.5%, 6.5% to 7%, 7% to 7.5%, 7.5% to 8%, 8% to 8.5%, 8.5% to 9%, 9% to 9.5%, 9.5% to 10%, 10% to 10.5%, 10.5% to 11%, 11% to 11.5%, 11.5% to 12%, 12% to 12.5%, 12.5% to 13%, 13% to 13.5%, 13.5% to 14%, 14% to 14.5%, 14.5% to 15%, 15% to 15.5%, 15.5% to 16%, 16% to 16.5%, 16.5% to 17%, 17% to 17.5%, 17.5% to 18%, 18% to 18.5%, 18.5% to 19%, 19% to 19.5% and 19.5% to 20%.

In certain embodiments, the amount of isotonic agent/cosolvent in pharmaceutical composition used is in the range of 0.01% to 0.05%, 0.05% to 0.1%, 0.1% to 0.15%, 0.15% to 0.2%, 0.2% to 0.25%, 0.25% to 0.3%, 0.3% to 0.35%, 0.35% to 0.4%, 0.4% to 0.45%, 0.45% to 0.5%, 0.5% to 0.55%, 0.55% to 0.6%, 0.6% to 0.65%, 0.65% to 0.7%, 0.7% to 0.75%, 0.75% to 0.8%, 0.8% to 0.85%, 0.85% to 0.9%, 0.9% to 0.95%, 0.95% to 1%, 1% to 1.05%, 1.05% to 1.1%, 1.1% to 1.15%, 1.15% to 1.2%, 1.2% to 1.25%, 1.25% to 1.3%, 1.3% to 1.35%, 1.35% to 1.4%, 1.4% to 1.45%, 1.45% to 1.5%, 1.5% to 1.55%, 1.55% to 1.6%, 1.6% to 1.65%, 1.65% to 1.7%, 1.7% to 1.75%, 1.75% to 1.8%, 1.8% to 1.85%, 1.85% to 1.9%, 1.9% to 1.95%, 1.95% to 2%, 2% to 2.15%, 2.15% to 2.2%, 2.2% to 2.25%, 2.25% to 2.3%, 2.3% to 2.35%, 2.35% to 2.4%, 2.4% to 2.45%, 2.45% to 2.5%, 2.5% to 2.55%, 2.55% to 2.6%, 2.6% to 2.65%, 2.65% to 2.7%, 2.7% to 2.75%, 2.75% to 2.8%, 2.8% to 2.85%, 2.85% to 2.9%, 2.9% to 2.95%, 2.95% to 3%, 3% to 3.05%, 3.05% to 3.1%, 3.1% to 3.15%, 3.15% to 3.2%, 3.2% to 3.25%, 3.25% to 3.3%, 3.3% to 3.35%, 3.35% to 3.4%, 3.4% to 3.45%, 3.45% to 3.5%, 3.5% to 3.55%, 3.55% to 3.6%, 3.6% to 3.65%, 3.65% to 3.7%, 3.7% to 3.75%, 3.75% to 3.8%, 3.8% to 3.85%, 3.85% to 3.9%, 3.9% to 3.95%, 3.95% to 4%, 4% to 4.05%, 4.05% to 4.1%, 4.1% to 4.15%, 4.15% to 4.2%, 4.2% to 4.25%, 4.25% to 4.3%, 4.3% to 4.35%, 4.35% to 4.4%, 4.4% to 4.45%, 4.45% to 4.5%, 4.5% to 4.55%, 4.55% to 4.6%, 4.6% to 4.65%, 4.65% to 4.7%, 4.7% to 4.75%, 4.75% to 4.8%, 4.8% to 4.85%, 4.85% to 4.9%, 4.9% to 4.95% and 4.95% to 5%.

In certain embodiments, the amount of isotonic agent/cosolvent in pharmaceutical composition used is in the range of 0.1% to 1.5%.

An alkalizing agent may be selected from but is not limited to Triethanolamine (Trolamine), Calcium Hydroxide, Diethanolamine, Ethanolamine (Monoethanolamine), Potassium Hydroxide, Sodium Carbonate, Sodium Citrate, Sodium Hydroxide, Meglumine, Sodium lactate, Tromethamine or suitable mixture thereof.

In an aspect, the present invention relates to a parenteral composition of Desidustat or pharmaceutically acceptable salts thereof wherein an amount of alkalizing agent used is in the range of 0.001% to 20% w/v.

Further, amount of alkalizing agent in pharmaceutical composition used is in the range of 0.001% to 0.005%, 0.005% to 0.01%, 0.01% to 0.02%, 0.02% to 0.03%, 0.03% to 0.04%, 0.04% to 0.05%, 0.05% to 0.06%, 0.06% to 0.07%, 0.07% to 0.08%, 0.08% to 0.09%, 0.09% to 0.1%, 0.1% to 0.5%, 0.5% to 1%, 1% to 1.5%, 1.5% to 2%, 2% to 2.5%, 2.5% to 3%, 3% to 3.5%, 3.5% to 4%, 4% to 4.5%, 4.5% to 5%, 5% to 5.5%, 5.5% to 6%, 6% to 6.5%, 6.5% to 7%, 7% to 7.5%, 7.5% to 8%, 8% to 8.5%, 8.5% to 9%, 9% to 9.5%, 9.5% to 10%, 10% to 10.5%, 10.5% to 11%, 11% to 11.5%, 11.5% to 12%, 12% to 12.5%, 12.5% to 13%, 13% to 13.5%, 13.5% to 14%, 14% to 14.5%, 14.5% to 15%, 15% to 15.5%, 15.5% to 16%, 16% to 16.5%, 16.5% to 17%, 17% to 17.5%, 17.5% to 18%, 18% to 18.5%, 18.5% to 19%, 19% to 19.5% and 19.5% to 20%.

In certain embodiments, the amount of alkalizing agent used in the pharmaceutical composition is in the range of 0.01% to 0.05%, 0.05% to 0.1%, 0.1% to 0.15%, 0.15% to 0.2%, 0.2% to 0.25%, 0.25% to 0.3%, 0.3% to 0.35%, 0.35% to 0.4%, 0.4% to 0.45%, 0.45% to 0.5%, 0.5% to 0.55%, 0.55% to 0.6%, 0.6% to 0.65%, 0.65% to 0.7%, 0.7% to 0.75%, 0.75% to 0.8%, 0.8% to 0.85%, 0.85% to 0.9%, 0.9% to 0.95%, 0.95% to 1%, 1% to 1.05%, 1.05% to 1.1%, 1.1% to 1.15%, 1.15% to 1.2%, 1.2% to 1.25%, 1.25% to 1.3%, 1.3% to 1.35%, 1.35% to 1.4%, 1.4% to 1.45%, 1.45% to 1.5%, 1.5% to 1.55%, 1.55% to 1.6%, 1.6% to 1.65%, 1.65% to 1.7%, 1.7% to 1.75%, 1.75% to 1.8%, 1.8% to 1.85%, 1.85% to 1.9%, 1.9% to 1.95%, 1.95% to 2%, 2% to 2.15%, 2.15% to 2.2%, 2.2% to 2.25%, 2.25% to 2.3%, 2.3% to 2.35%, 2.35% to 2.4%, 2.4% to 2.45%, 2.45% to 2.5%, 2.5% to 2.55%, 2.55% to 2.6%, 2.6% to 2.65%, 2.65% to 2.7%, 2.7% to 2.75%, 2.75% to 2.8%, 2.8% to 2.85%, 2.85% to 2.9%, 2.9% to 2.95%, 2.95% to 3%, 3% to 3.05%, 3.05% to 3.1%, 3.1% to 3.15%, 3.15% to 3.2%, 3.2% to 3.25%, 3.25% to 3.3%, 3.3% to 3.35%, 3.35% to 3.4%, 3.4% to 3.45%, 3.45% to 3.5%, 3.5% to 3.55%, 3.55% to 3.6%, 3.6% to 3.65%, 3.65% to 3.7%, 3.7% to 3.75%, 3.75% to 3.8%, 3.8% to 3.85%, 3.85% to 3.9%, 3.9% to 3.95%, 3.95% to 4%, 4% to 4.05%, 4.05% to 4.1%, 4.1% to 4.15%, 4.15% to 4.2%, 4.2% to 4.25%, 4.25% to 4.3%, 4.3% to 4.35%, 4.35% to 4.4%, 4.4% to 4.45%, 4.45% to 4.5%, 4.5% to 4.55%, 4.55% to 4.6%, 4.6% to 4.65%, 4.65% to 4.7%, 4.7% to 4.75%, 4.75% to 4.8%, 4.8% to 4.85%, 4.85% to 4.9%, 4.9% to 4.95% and 4.95% to 5%.

In certain embodiments, the amount of alkalizing agent in pharmaceutical composition used is in the range of 0.5% to 3%.

In an embodiment, parenteral pharmaceutical composition may use as Intramuscular, Subcutaneous, Intravenous. Parenteral solution may use as ready to use solution. Parenteral solution is in the form of injectable.

In certain embodiments, parenteral pharmaceutical composition of Desidustat or pharmaceutically acceptable salts thereof can be further characterized according to the dose of compound administered to a subject, where subject is animal or human.

In another embodiments, the parenteral pharmaceutical composition is administered to the subject once a day, twice a day or thrice a day or on alternate day for at least 2 weeks.

In a further embodiment, use of parenteral composition of Desidustat or pharmaceutically acceptable salts thereof in the manufacture of a medicament for treatment of radiation induced toxicity.

Method of Treating Radiation Induced Toxicity by Oral Composition of Desidustat or Pharmaceutically Acceptable Salts Thereof

In an embodiment, present invention provides a method of treating radiation induced toxicity in a subject by oral composition of Desidustat or pharmaceutically acceptable salts thereof optionally with one or more pharmaceutically acceptable excipients.

In an embodiment, radiation induced toxicity includes acute radiation syndrome.

In another embodiment, Acute Radiation Syndrome is selected from Hematopoietic Acute Radiation Syndrome (H-ARS).

Hematopoietic Acute Radiation Syndrome occurs when the whole body or a significant portion of body is exposed to a high dose of penetrating ionizing radiation, typically between 0.7 and 10 Gray (Gy), delivered over a short period (minutes to hours).

An oral composition of Desidustat is useful for the treatment of Hematopoietic Acute Radiation Syndrome.

The suitable pharmaceutically acceptable salts of Desidustat may be selected from suitable inorganic metal salt or an organic amine salt.

In certain embodiments, the inorganic metal salts can be selected from calcium salt, sodium salt, potassium salt, lithium salt, barium salt, strontium salt, magnesium salt, cesium salt, copper salt, cobalt salt, iron salt, manganese salt, lead salt, aluminium salt, cadmium salt, silver salt, and zinc salt, and the like.

In certain embodiments, the organic amine salts, also referred to herein as organic ammonium salts can be selected from methylammonium salt, dimethylammonium salt, ethylammonium salt, diethyl ammonium salt, n-propyl ammonium salt, isopropyl ammonium salt, diisopropyl ammonium salt, N-methyl isopropyl ammonium salt, n-butyl ammonium salt, t-butyl ammonium salt, 2-butammonium salt, 1,2-ethane diammonium salt, N-methylglucammonium salt, N,N,N-trimethyl ethanolammonium hydroxide (choline) salt, tromethammmonium salt, cyclohexylammonium salt, N-methyl cyclohexylammonium salt, guanidinium salt, N-(4-aminobutyl) guanidinium salt, dicyclohexylammonium salt, benzene-methanammonium salt, ethanolammonium salt, diethanolammonium salt, tris-(hydroxymethyl)methylammonium salt, hydroxylammonium salt, methanammonium salt, benzylammonium salt, N-methylbenzylammonium salt, N-ethyl benzylammonium salt, 4-methoxybenzylammonium salt, pyrrolidinium salt, piperidinium salt, piperazinium salt, morpholinium salt, 2-aminopyrimidinium salt, alaninium salt, lysinium salt, argininium salt, histidinium salt, threoninium salt, prolinium salt, glutammonium salt, glycinium salt, 2-thiopheneethanammonium salt, (2S)-3,3-dimethyl-2-butanammonium salt, cyclopentanammonium salt, cycloheptanammonium salt, meglummonium salt, bethammonium salt, dibenzylammonium salt, diphenylammonium salt, α-naphthylammonium salt, O-phenylenediammonium salt, 1,3-Diaminopropammonium salt, (S)-α-naphthylethylammonium salt, (S)-3-methoxyphenylethylammonium salt, (S)-4-methoxyphenylethylammonium salt, (S)-4-chlorophenylethylammonium salt, (S)-4-methylphenylethylammonium salt, cinchoninium salt, cinchonidinium salt, (−)-quininium salt, triethanolammonium salt, imidazolium salt, ethylenediammonium salt, polammonium salt, morpholinium 4-(2-hydroxyethyl) salt, N—N-diethylethanolammonium salt, deammonium salt, hydrabammonium salt, betammonium salt, adamantanammonium salt, L-adamantanmethylammonium salt, tritylammonium salt, glucammonium salt, N-methyl pyrrolidinium salt, ureammonium salt, procammonium salt, metformmonium salt, hexane1-6-diammonium salt, 2-(2-aminoethoxy)ethanammonium salt, N-methylmorpholinium salt, and N-ethylmorpholinium salt.

In an another embodiment, the uncoated tablet comprises concentration of Desidustat in the range of 1% w/w to 90% w/w, dose of Desidustat in the range of 1% w/w to 80% w/w, dose of Desidustat in the range of 1% w/w to 70% w/w, dose of Desidustat in the range of 1% w/w to 60% w/w, dose of Desidustat in the range of 1% w/w to 50% w/w, dose of Desidustat in the range of 1% w/w to 40% w/w, dose of Desidustat in the range of 1% w/w to 30% w/w, dose of Desidustat in the range of 1% w/w to 20% w/w or dose of Desidustat in the range of 1% w/w to 10% w/w.

In one embodiment, the present invention provides an uncoated tablet comprises Desidustat or pharmaceutically acceptable salts thereof and one or more a pharmaceutically acceptable excipient.

In an embodiment pharmaceutical excipient according to present invention can be selected from diluents or fillers, disintegrants, binder, lubricants, glidants, film forming agents, plasticizers, opacifier, solvents, and the like as known in the art.

As used herein the term “diluent or filler” refers to substances that are used to dilute the active pharmaceutical ingredient prior to delivery. Diluents can also serve as stabilizers.

Non-limiting examples of diluents include starch and its processed and co-processed derivatives, saccharides, di saccharides, sucrose, lactose, polysaccharides, cellulose, cellulose ethers, cellulose acetate, hydroxypropyl cellulose, sugar alcohols, xylitol, sorbitol, maltitol, lactitol, microcrystalline cellulose, magnesium or calcium or sodium carbonate, lactose, lactose monohydrate, di-calcium phosphate, compressible sugars, di basic calcium phosphate dihydrate, mannitol lactose anhydrous, magnesium oxide, maltodextrin, maltose, pullulan, sodium alginate, sodium bicarbonate, calcium silicate, calcium sulphate, cell and tribasic calcium phosphate or suitable combinations thereof.

As used herein the term “disintegrant or disintegrating agents” refers to a substance which, upon addition to a solid preparation, facilitates its break-up or disintegration after administration and permits the release of an active ingredient as efficiently as possible to allow for its rapid dissolution. Non-limiting examples of disintegrants include maize starch, sodium starch glycolate, croscarmellose sodium, crospovidone, microcrystalline cellulose, modified corn starch, sodium carboxymethyl starch, povidone, pregelatinized starch, agar, carboxymethyl cellulose calcium or sodium, colloidal silicon dioxide, chitosan, docusate sodium, hydroxyl propyl cellulose, magnesium aluminium silicate, maltose, methyl cellulose, polacrilin potassium, and alginic acid or suitable combinations thereof.

As used herein the term “binder” refers to any pharmaceutically acceptable substance which can be used to bind the active and inert components together to maintain cohesive and discrete portions. Non-limiting examples of binders chitosan, hydrogenated castor oil, sodium alginate, carbomers, cellulose acetate phthalate, povidone, sugar, hydroxypropylmethyl-cellulose, hydroxypropylcellulose, starch, alginic acid, pregelatinized starch, acacia, tragakanth, ethylcellulose, acrylic and methacrylic acid co polymers or suitable combinations thereof.

As used herein the term “lubricant” refers to an excipient, which is added to a powder blend to prevent the compacted powder mass from sticking to the equipment during the tableting or encapsulation process. It aids the ejection of the tablet form the dies, and can improve powder flow. Non-limiting examples of lubricants include magnesium stearate, stearic acid, silica, fats, zinc or sucrose or sodium or calcium stearate, castor oil, hydrogenated castor oil, polyethylene glycol and its derivatives, sodium stearyl fumarate, talc, or fatty acids including lauric acid, oleic acid, glyceryl behenate, glyceryl monostearate, and C₁-C₁₀ fatty acid or suitable combinations thereof.

As used herein the term “glidant” is intended to mean agents used in tablet and capsule formulations to improve flow-properties during tablet compression and to produce an anti caking effect. Non-limiting examples of glidants include colloidal silicon dioxide, talc, fumed silica, starch, starch derivatives, and bentonite or suitable combinations thereof.

As used herein the term “film forming agents” refers to but not limited to hydroxypropyl methylcellulose, methylcellulose, ethylcellulose, hydroxypropyl cellulose, povidone, polydextrose, lactose, maltodextrin, acrylic polymer or suitable combinations thereof.

As used herein the term “plasticizer” refers to but not limited to polyols like polyethylene glycols, propylene glycol, glycerol (glycerine), organic esters like phthalate esters (diethyl, dibutyl), dibutyl sebacete, citrate esters (triethyl, acetyl triethyl, acetyl tributyl), triacetin, Oils/glycerides like castor oil; acetylated monoglycerides, fractionated coconut oil or suitable combinations thereof.

As used herein the term “opacifier” refers to but not limited to titanium dioxide, talc, sunset yellow, tartrazine, erythrosine, iron oxide yellow, red and black, carmine, anthocyanins. allura Red AC, allura Red AC aluminum lake, indigotine, indigotine aluminum lake or suitable combinations thereof.

One or more “solvents” used in the formulation are selected from water, acetone, chloroform, dichlorome thane, ethyl alcohol, ethyl acetate, methyl alcohol, isopropyl alcohol, N,N-dimethyl formamide and combinations thereof and other such materials known to those of ordinary skill in the art.

In an another embodiment, the uncoated tablet comprises from about 1% w/w to about 90% w/w Desidustat, microcrystalline cellulose from about 2% w/w to about 90% w/w, croscarmellose sodium from about 0.5% w/w to 10% w/w, lactose monohydrate from about 2% w/w to about 90% w/w, hypromellose 3 cps from about 0.5% w/w to about 10% w/w, Talc from about 0.5% w/w to about 3% w/w, magnesium Stearate from about 0.5% w/w to about 5% w/w of the total composition, polyvinyl pyrrolidone from about 0.5% w/w to about 10% w/w, starch from about 1% w/w to about 20% w/w based on the weight of uncoated tablet.

In certain embodiments, oral pharmaceutical composition of Desidustat or pharmaceutically acceptable salts thereof can be further characterized according to the dose of compound administered to a subject, where subject is animal or human.

In another embodiment, the oral pharmaceutical composition is administered to the subject once a day, twice a day or thrice a day or on alternate day for at least 2 weeks.

In a further embodiment, use of oral composition of Desidustat or pharmaceutically acceptable salts thereof in the manufacture of a medicament for treatment of radiation induced toxicity.

The method may be further characterized according to the treatment increases hemoglobin and/or red blood cell (RBC) count relative to baseline of a subject due to the therapeutic method (e.g., administering Desidustat or a pharmaceutically acceptable salt thereof to the subject). For example, in certain embodiments, the method increases hemoglobin and/or red blood cell (RBC) count relative to baseline of the subject. In certain embodiments, the treatment increases hemoglobin and/or red blood cell (RBC) count relative to baseline of the subject by at least 5%. In certain embodiments, the treatment increases hemoglobin and/or red blood cell (RBC) count relative to baseline of the subject by at least 10%. In certain embodiments, the treatment increases hemoglobin and/or red blood cell (RBC) count relative to baseline of the subject by at least 15%. In certain embodiments, the treatment increases hemoglobin and/or red blood cell (RBC) count relative to baseline of the subject by at least 20%. In certain embodiments, the treatment increases hemoglobin and/or red blood cell (RBC) count relative to baseline of the subject by at least 25%. In certain embodiments, the treatment increases hemoglobin and/or red blood cell (RBC) count relative to baseline of the subject by at least 30%. In certain embodiments, the treatment increases hemoglobin and/or red blood cell (RBC) count relative to baseline of the subject by at least 35%. In certain embodiments, the treatment increases hemoglobin and/or red blood cell (RBC) count relative to baseline of the subject by at least 40%. In certain embodiments, the treatment increases hemoglobin and/or red blood cell (RBC) count relative to baseline of the subject by at least 45%. In certain embodiments, the treatment increases hemoglobin and/or red blood cell (RBC) count relative to baseline of the subject by at least 50%.

### Use of Desidustat or Pharmaceutically Acceptable Salts Thereof for the Treatment of Radiation Induced Toxicity

In an embodiment the present invention provides a use of Desidustat or pharmaceutically acceptable salts thereof in a subject, for the treatment of radiation induced toxicity.

In an embodiment, radiation induced toxicity includes acute radiation syndrome.

In another embodiment, Acute Radiation Syndrome is selected from Hematopoietic Acute Radiation Syndrome (H-ARS).

Hematopoietic Acute Radiation Syndrome occurs when the whole body or a significant portion of body is exposed to a high dose of penetrating ionizing radiation, typically between 0.7 and 10 Gray (Gy), delivered over a short period (minutes to hours).

Desidustat is useful for the treatment of Hematopoietic Acute Radiation Syndrome.

The suitable pharmaceutically acceptable salts of Desidustat may be selected from suitable inorganic metal salt or an organic amine salt.

In certain embodiments, the inorganic metal salts can be selected from calcium salt, sodium salt, potassium salt, lithium salt, barium salt, strontium salt, magnesium salt, cesium salt, copper salt, cobalt salt, iron salt, manganese salt, lead salt, aluminium salt, cadmium salt, silver salt, and zinc salt, and the like.

In certain embodiments, the organic amine salts, also referred to herein as organic ammonium salts can be selected from methylammonium salt, dimethylammonium salt, ethylammonium salt, diethyl ammonium salt, n-propyl ammonium salt, isopropyl ammonium salt, diisopropyl ammonium salt, N-methyl isopropyl ammonium salt, n-butyl ammonium salt, t-butyl ammonium salt, 2-butammonium salt, 1,2-ethane diammonium salt, N-methylglucammonium salt, N,N,N-trimethyl ethanolammonium hydroxide (choline) salt, tromethammmonium salt, cyclohexylammonium salt, N-methyl cyclohexylammonium salt, guanidinium salt, N-(4-aminobutyl) guanidinium salt, dicyclohexylammonium salt, benzene-methanammonium salt, ethanolammonium salt, diethanolammonium salt, tris-(hydroxymethyl)methylammonium salt, hydroxylammonium salt, methanammonium salt, benzylammonium salt, N-methylbenzylammonium salt, N-ethyl benzylammonium salt, 4-methoxybenzylammonium salt, pyrrolidinium salt, piperidinium salt, piperazinium salt, morpholinium salt, 2-aminopyrimidinium salt, alaninium salt, lysinium salt, argininium salt, histidinium salt, threoninium salt, prolinium salt, glutammonium salt, glycinium salt, 2-thiopheneethanammonium salt, (2S)-3,3-dimethyl-2-butanammonium salt, cyclopentanammonium salt, cycloheptanammonium salt, meglummonium salt, bethammonium salt, dibenzylammonium salt, diphenylammonium salt, α-naphthylammonium salt, O-phenylenediammonium salt, 1,3-Diaminopropammonium salt, (S)-α-naphthylethylammonium salt, (S)-3-methoxyphenylethylammonium salt, (S)-4-methoxyphenylethylammonium salt, (S)-4-chlorophenylethylammonium salt, (S)-4-methylphenylethylammonium salt, cinchoninium salt, cinchonidinium salt, (−)-quininium salt, triethanolammonium salt, imidazolium salt, ethylenediammonium salt, polammonium salt, morpholinium 4-(2-hydroxyethyl) salt, N—N-diethylethanolammonium salt, deammonium salt, hydrabammonium salt, betammonium salt, adamantanammonium salt, L-adamantanmethylammonium salt, tritylammonium salt, glucammonium salt, N-methyl pyrrolidinium salt, ureammonium salt, procammonium salt, metformmonium salt, hexane1-6-diammonium salt, 2-(2-aminoethoxy)ethanammonium salt, N-methylmorpholinium salt, and N-ethylmorpholinium salt.

In certain embodiments, effective amount of Desidustat or pharmaceutically acceptable salts thereof, selected from 1 mg to 500 mg. In certain embodiments, effective amount of Desidustat or pharmaceutically acceptable salts thereof, selected from 1 mg to 400 mg. In certain embodiments, effective amount of Desidustat or pharmaceutically acceptable salts thereof, selected from 1 mg to 300 mg. In certain embodiments, effective amount of Desidustat or pharmaceutically acceptable salts thereof, selected from 1 mg to 250 mg. In certain embodiments, effective amount of Desidustat or pharmaceutically acceptable salts thereof, selected from 1 mg to 200 mg. In certain embodiments, effective amount of Desidustat or pharmaceutically acceptable salts thereof, selected from 1 mg to 150 mg. In certain embodiments, effective amount of Desidustat or pharmaceutically acceptable salts thereof, selected from 1 mg to 100 mg. In certain embodiments, effective amount of Desidustat or pharmaceutically acceptable salts thereof, selected from 1 mg to 75 mg. In certain embodiments, Desidustat or pharmaceutically acceptable salts thereof, in a concentration selected from 1 mg to 50 mg In certain embodiments, effective amount of Desidustat or pharmaceutically acceptable salts thereof, selected from 1 mg to 25 mg. In certain embodiments, effective amount of Desidustat or pharmaceutically acceptable salts thereof, selected from 25 mg, 50 mg and 100 mg.

In certain embodiments, effective amount of Desidustat or pharmaceutically acceptable salts thereof, selected from 1 mg to 10 mg, 10 mg to 20 mg, 20 mg to 30 mg, 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 110 mg, 110 mg to 120 mg, 120 mg to 130 mg, 130 mg to 140 mg, 140 mg to 150 mg, 150 mg to 160 mg, 160 mg to 170 mg, 170 mg to 180 mg, 180 mg to 190 mg, 190 mg to 200 mg, 200 mg to 210 mg, 210 mg to 220 mg, 220 mg to 230 mg, 230 mg to 240 mg and 240 mg to 250 mg.

In certain embodiments, effective amount of Desidustat or pharmaceutically acceptable salts thereof, selected from 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, and 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg and 150 mg.

In certain embodiments, Desidustat or pharmaceutically acceptable salts thereof can be further characterized according to the dose of compound administered to a subject, where subject is animal or human.

In another embodiments, Desidustat is administered to the subject once a day, twice a day or thrice a day or on alternate day for at least 2 weeks.

In a further embodiment, the present invention provides effective amount of Desidustat or its pharmaceutically acceptable salt is administered to subject by oral pharmaceutical composition or parenteral pharmaceutical composition.

### Use of Parenteral Pharmaceutical Composition of Desidustat or Pharmaceutically Acceptable Salts Thereof for the Treatment of Radiation Induced Toxicity

In an embodiment, present invention provides a use of parenteral pharmaceutical composition of Desidustat or pharmaceutically acceptable salts thereof optionally with one or more pharmaceutically acceptable excipients for the treatment of radiation induced toxicity.

In an embodiment, radiation induced toxicity includes acute radiation syndrome.

In another embodiment, Acute Radiation Syndrome is selected from Hematopoietic Acute Radiation Syndrome (H-ARS), Gastrointestinal Syndrome and Cardiovascular/Central Nervous System Syndrome.

Hematopoietic Acute Radiation Syndrome occurs when the whole body or a significant portion of body is exposed to a high dose of penetrating ionizing radiation, typically between 0.7 and 10 Gray (Gy), delivered over a short period (minutes to hours).

The parenteral pharmaceutical composition of Desidustat is useful for the treatment of Hematopoietic Acute Radiation Syndrome.

The suitable pharmaceutically acceptable salts of Desidustat may be selected from suitable inorganic metal salt or an organic amine salt.

In certain embodiments, the inorganic metal salts can be selected from calcium salt, sodium salt, potassium salt, lithium salt, barium salt, strontium salt, magnesium salt, cesium salt, copper salt, cobalt salt, iron salt, manganese salt, lead salt, aluminium salt, cadmium salt, silver salt, and zinc salt, and the like.

In certain embodiments, the organic amine salts, also referred to herein as organic ammonium salts can be selected from methylammonium salt, dimethylammonium salt, ethylammonium salt, diethyl ammonium salt, n-propyl ammonium salt, isopropyl ammonium salt, diisopropyl ammonium salt, N-methyl isopropyl ammonium salt, n-butyl ammonium salt, t-butyl ammonium salt, 2-butammonium salt, 1,2-ethane diammonium salt, N-methylglucammonium salt, N,N,N-trimethyl ethanolammonium hydroxide (choline) salt, tromethammmonium salt, cyclohexylammonium salt, N-methyl cyclohexylammonium salt, guanidinium salt, N-(4-aminobutyl) guanidinium salt, dicyclohexylammonium salt, benzene-methanammonium salt, ethanolammonium salt, diethanolammonium salt, tris-(hydroxymethyl)methylammonium salt, hydroxylammonium salt, methanammonium salt, benzylammonium salt, N-methylbenzylammonium salt, N-ethyl benzylammonium salt, 4-methoxybenzylammonium salt, pyrrolidinium salt, piperidinium salt, piperazinium salt, morpholinium salt, 2-aminopyrimidinium salt, alaninium salt, lysinium salt, argininium salt, histidinium salt, threoninium salt, prolinium salt, glutammonium salt, glycinium salt, 2-thiopheneethanammonium salt, (2S)-3,3-dimethyl-2-butanammonium salt, cyclopentanammonium salt, cycloheptanammonium salt, meglummonium salt, bethammonium salt, dibenzylammonium salt, diphenylammonium salt, α-naphthylammonium salt, O-phenylenediammonium salt, 1,3-Diaminopropammonium salt, (S)-α-naphthylethylammonium salt, (S)-3-methoxyphenylethylammonium salt, (S)-4-methoxyphenylethylammonium salt, (S)-4-chlorophenylethylammonium salt, (S)-4-methylphenylethylammonium salt, cinchoninium salt, cinchonidinium salt, (−)-quininium salt, triethanolammonium salt, imidazolium salt, ethylenediammonium salt, polammonium salt, morpholinium 4-(2-hydroxyethyl) salt, N—N-diethylethanolammonium salt, deammonium salt, hydrabammonium salt, betammonium salt, adamantanammonium salt, L-adamantanmethylammonium salt, tritylammonium salt, glucammonium salt, N-methyl pyrrolidinium salt, ureammonium salt, procammonium salt, metformmonium salt, hexane1-6-diammonium salt, 2-(2-aminoethoxy)ethanammonium salt, N-methylmorpholinium salt, and N-ethylmorpholinium salt.

In certain embodiments, Desidustat or pharmaceutically acceptable salts thereof, in a concentration selected from 1 mg/mL to 250 mg/mL. In certain embodiments, Desidustat or pharmaceutically acceptable salts thereof, in a concentration selected from 1 mg/mL to 200 mg/mL. In certain embodiments, Desidustat or pharmaceutically acceptable salts thereof, in a concentration selected from 1 mg/mL to 150 mg/mL. In certain embodiments, Desidustat or pharmaceutically acceptable salts thereof, in a concentration selected from 1 mg/mL to 125 mg/mL. In certain embodiments, Desidustat or pharmaceutically acceptable salts thereof, in a concentration selected from 1 mg/mL to 100 mg/mL. In certain embodiments, Desidustat or pharmaceutically acceptable salts thereof, in a concentration selected from 1 mg/mL to 75 mg/mL. In certain embodiments, Desidustat or pharmaceutically acceptable salts thereof, in a concentration selected from 1 mg/mL to 50 mg/mL. In certain embodiments, Desidustat or pharmaceutically acceptable salts thereof, in a concentration selected from 1 mg/mL to 25 mg/mL. In certain embodiments, Desidustat or pharmaceutically acceptable salts thereof, in a concentration selected from 1 mg/mL to 10 mg/mL. In certain embodiments, Desidustat or pharmaceutically acceptable salts thereof in a concentration selected from 25 mg/mL, 40 mg/mL, 50 mg/mL and 100 mg/mL.

Further, concentration of Desidustat or pharmaceutically acceptable salts thereof in parenteral pharmaceutical composition is selected from 1 mg/mL to 10 mg/mL, 10 mg/mL to 20 mg/mL, 20 mg/mL to 30 mg/mL, 30 mg/mL to 40 mg/mL, 40 mg/mL to 50 mg/mL, 50 mg/mL to 60 mg/mL, 60 mg/mL to 70 mg/mL, 70 mg/mL to 80 mg/mL, 80 mg/mL to 90 mg/mL, 90 mg/mL to 100 mg/mL, 100 mg/mL to 110 mg/mL, 110 mg/mL to 120 mg/mL, 120 mg/mL to 130 mg/mL, 130 mg/mL to 140 mg/mL, 140 mg/mL to 150 mg/mL, 150 mg/mL to 160 mg/mL, 160 mg/mL to 170 mg/mL, 170 mg/mL to 180 mg/mL, 180 mg/mL to 190 mg/mL, 190 mg/mL to 200 mg/mL, 200 mg/mL to 210 mg/mL, 210 mg/mL to 220 mg/mL, 220 mg/mL to 230 mg/mL, 230 mg/mL to 240 mg/mL and 240 mg/mL to 250 mg/mL.

Preferably, concentration of Desidustat or pharmaceutically acceptable salts thereof in parenteral pharmaceutical composition is selected from 1 mg/mL to 2 mg/mL, 2 mg/mL to 3 mg/mL, 3 mg/mL to 4 mg/mL, 4 mg/mL to 5 mg/mL, 5 mg/mL to 6 mg/mL, 6 mg/mL to 7 mg/mL, 7 mg/mL to 8 mg/mL, 8 mg/mL to 9 mg/mL, 9 mg/mL to 10 mg/mL, 10 mg/mL to 11 mg/mL, 11 mg/mL to 12 mg/mL, 12 mg/mL to 13 mg/mL, 13 mg/mL to 14 mg/mL, 14 mg/mL to 15 mg/mL, 15 mg/mL to 16 mg/mL, 16 mg/mL to 17 mg/mL, 17 mg/mL to 18 mg/mL, 18 mg/mL to 19 mg/mL, 19 mg/mL to 20 mg/mL, 20 mg/mL to 21 mg/mL, 21 mg/mL to 22 mg/mL, 22 mg/mL to 23 mg/mL, 23 mg/mL to 24 mg/mL, 24 mg/mL to 25 mg/mL, 25 mg/mL to 26 mg/mL, 26 mg/mL to 27 mg/mL, 27 mg/mL to 28 mg/mL, 28 mg/mL to 29 mg/mL, 29 mg/mL to 30 mg/mL, 30 mg/mL to 31 mg/mL, 31 mg/mL to 32 mg/mL, 32 mg/mL to 33 mg/mL, 33 mg/mL to 34 mg/mL, 34 mg/mL to 35 mg/mL, 35 mg/mL to 36 mg/mL, 36 mg/mL to 37 mg/mL, 37 mg/mL to 38 mg/mL, 38 mg/mL to 39 mg/mL, 39 mg/mL to 40 mg/mL, 40 mg/mL to 41 mg/mL, 41 mg/mL to 42 mg/mL, 42 mg/mL to 43 mg/mL, 43 mg/mL to 44 mg/mL, 44 mg/mL to 45 mg/mL, 45 mg/mL to 46 mg/mL, 46 mg/mL to 47 mg/mL, 47 mg/mL to 48 mg/mL, 48 mg/mL to 49 mg/mL, 49 mg/mL to 50 mg/mL, 50 mg/mL to 51 mg/mL, 51 mg/mL to 52 mg/mL, 52 mg/mL to 53 mg/mL, 53 mg/mL to 54 mg/mL, 54 mg/mL to 55 mg/mL, 55 mg/mL to 56 mg/mL, 56 mg/mL to 57 mg/mL, 57 mg/mL to 58 mg/mL, 58 mg/mL to 59 mg/mL, 59 mg/mL to 60 mg/mL, 60 mg/mL to 61 mg/mL, 61 mg/mL to 62 mg/mL, 62 mg/mL to 63 mg/mL, 63 mg/mL to 64 mg/mL, 64 mg/mL to 65 mg/mL, 65 mg/mL to 66 mg/mL, 66 mg/mL to 67 mg/mL, 67 mg/mL to 68 mg/mL, 68 mg/mL to 69 mg/mL, 69 mg/mL to 70 mg/mL, 70 mg/mL to 71 mg/mL, 71 mg/mL to 72 mg/mL, 72 mg/mL to 73 mg/mL, 73 mg/mL to 74 mg/mL, 74 mg/mL to 75 mg/mL, 75 mg/mL to 76 mg/mL, 76 mg/mL to 77 mg/mL, 77 mg/mL to 78 mg/mL, 78 mg/mL to 79 mg/mL, 79 mg/mL to 80 mg/mL, 80 mg/mL to 81 mg/mL, 81 mg/mL to 82 mg/mL, 82 mg/mL to 83 mg/mL, 83 mg/mL to 84 mg/mL, 84 mg/mL to 85 mg/mL, 85 mg/mL to 86 mg/mL, 86 mg/mL to 87 mg/mL, 87 mg/mL to 88 mg/mL, 88 mg/mL to 89 mg/mL, 89 mg/mL to 90 mg/mL, 90 mg/mL to 91 mg/mL, 91 mg/mL to 92 mg/mL, 92 mg/mL to 93 mg/mL, 93 mg/mL to 94 mg/mL, 94 mg/mL to 95 mg/mL, 95 mg/mL to 96 mg/mL, 96 mg/mL to 97 mg/mL, 97 mg/mL to 98 mg/mL, 98 mg/mL to 99 mg/mL, 99 mg/mL to 100 mg/mL, 100 mg/mL to 101 mg/mL, 101 mg/mL to 102 mg/mL, 102 mg/mL to 103 mg/mL, 103 mg/mL to 104 mg/mL, 104 mg/mL to 105 mg/mL, 105 mg/mL to 106 mg/mL, 106 mg/mL to 107 mg/mL, 107 mg/mL to 108 mg/mL, 108 mg/mL to 109 mg/mL, 109 mg/mL to 110 mg/mL, 110 mg/mL to 111 mg/mL, 111 mg/mL to 112 mg/mL, 112 mg/mL to 113 mg/mL, 113 mg/mL to 114 mg/mL, 114 mg/mL to 115 mg/mL, 115 mg/mL to 116 mg/mL, 116 mg/mL to 117 mg/mL, 117 mg/mL to 118 mg/mL, 118 mg/mL to 119 mg/mL, 119 mg/mL to 120 mg/mL, 120 mg/mL to 121 mg/mL, 121 mg/mL to 122 mg/mL, 122 mg/mL to 123 mg/mL, 123 mg/mL to 124 mg/mL, 124 mg/mL to 125 mg/mL, 125 mg/mL to 126 mg/mL, 126 mg/mL to 127 mg/mL, 127 mg/mL to 128 mg/mL, 128 mg/mL to 129 mg/mL, 129 mg/mL to 130 mg/mL, 130 mg/mL to 131 mg/mL, 131 mg/mL to 132 mg/mL, 132 mg/mL to 133 mg/mL, 133 mg/mL to 134 mg/mL, 134 mg/mL to 135 mg/mL, 135 mg/mL to 136 mg/mL, 136 mg/mL to 137 mg/mL, 137 mg/mL to 138 mg/mL, 138 mg/mL to 139 mg/mL, 139 mg/mL to 140 mg/mL, 140 mg/mL to 141 mg/mL, 141 mg/mL to 142 mg/mL, 142 mg/mL to 143 mg/mL, 143 mg/mL to 144 mg/mL, 144 mg/mL to 145 mg/mL, 145 mg/mL to 146 mg/mL, 146 mg/mL to 147 mg/mL, 147 mg/mL to 148 mg/mL, 148 mg/mL to 149 mg/mL and 149 mg/mL to 150 mg/mL.

More preferably, concentration of Desidustat or pharmaceutically acceptable salts thereof in parenteral pharmaceutical composition is selected from 1 mg/mL to 1.5 mg/mL, 1.5 mg/mL to 2 mg/mL, 2 mg/mL to 2.5 mg/mL, 2.5 mg/mL to 3 mg/mL, 3 mg/mL to 3.5 mg/mL, 3.5 mg/mL to 4 mg/mL, 4 mg/mL to 4.5 mg/mL, 4.5 mg/mL to 5 mg/mL, 5 mg/mL to 5.5 mg/mL, 5.5 mg/mL to 6 mg/mL, 6 mg/mL to 6.5 mg/mL, 6.5 mg/mL to 7 mg/mL, 7 mg/mL to 7.5 mg/mL, 7.5 mg/mL to 8 mg/mL, 8 mg/mL to 8.5 mg/mL, 8.5 mg/mL to 9 mg/mL, 9 mg/mL to 9.5 mg/mL, 9.5 mg/mL to 10 mg/mL, 10 mg/mL to 10.5 mg/mL, 10.5 mg/mL to 11 mg/mL, 11 mg/mL to 11.5 mg/mL, 11.5 mg/mL to 12 mg/mL, 12 mg/mL to 12.5 mg/mL, 12.5 mg/mL to 13 mg/mL, 13 mg/mL to 13.5 mg/mL, 13.5 mg/mL to 14 mg/mL, 14 mg/mL to 14.5 mg/mL, 14.5 mg/mL to 15 mg/mL, 15 mg/mL to 15.5 mg/mL, 15.5 mg/mL to 16 mg/mL, 16 mg/mL to 16.5 mg/mL, 16.5 mg/mL to 17 mg/mL, 17 mg/mL to 17.5 mg/mL, 17.5 mg/mL to 18 mg/mL, 18 mg/mL to 18.5 mg/mL, 18.5 mg/mL to 19 mg/mL, 19 mg/mL to 19.5 mg/mL, 19.5 mg/mL to 20 mg/mL, 20 mg/mL to 20.5 mg/mL, 20.5 mg/mL to 21 mg/mL, 21 mg/mL to 21.5 mg/mL, 21.5 mg/mL to 22 mg/mL, 22 mg/mL to 22.5 mg/mL, 22.5 mg/mL to 23 mg/mL, 23 mg/mL to 23.5 mg/mL, 23.5 mg/mL to 24 mg/mL, 24 mg/mL to 24.5 mg/mL, 24.5 mg/mL to 25 mg/mL, 25 mg/mL to 25.5 mg/mL, 25.5 mg/mL to 26 mg/mL, 26 mg/mL to 26.5 mg/mL, 26.5 mg/mL to 27 mg/mL, 27 mg/mL to 27.5 mg/mL, 27.5 mg/mL to 28 mg/mL, 28 mg/mL to 28.5 mg/mL, 28.5 mg/mL to 29 mg/mL, 29 mg/mL to 29.5 mg/mL, 29.5 mg/mL to 30 mg/mL, 30 mg/mL to 30.5 mg/mL, 30.5 mg/mL to 31 mg/mL, 31 mg/mL to 31.5 mg/mL, 31.5 mg/mL to 32 mg/mL, 32 mg/mL to 32.5 mg/mL, 32.5 mg/mL to 33 mg/mL, 33 mg/mL to 33.5 mg/mL, 33.5 mg/mL to 34 mg/mL, 34 mg/mL to 34.5 mg/mL, 34.5 mg/mL to 35 mg/mL, 35 mg/mL to 35.5 mg/mL, 35.5 mg/mL to 36 mg/mL, 36 mg/mL to 36.5 mg/mL, 36.5 mg/mL to 37 mg/mL, 37 mg/mL to 37.5 mg/mL, 37.5 mg/mL to 38 mg/mL, 38 mg/mL to 38.5 mg/mL, 38.5 mg/mL to 39 mg/mL, 39 mg/mL to 39.5 mg/mL, 39.5 mg/mL to 40 mg/mL, 40 mg/mL to 40.5 mg/mL, 40.5 mg/mL to 41 mg/mL, 41 mg/mL to 41.5 mg/mL, 41.5 mg/mL to 42 mg/mL, 42 mg/mL to 42.5 mg/mL, 42.5 mg/mL to 43 mg/mL, 43 mg/mL to 43.5 mg/mL, 43.5 mg/mL to 44 mg/mL, 44 mg/mL to 44.5 mg/mL, 44.5 mg/mL to 45 mg/mL, 45 mg/mL to 45.5 mg/mL, 45.5 mg/mL to 46 mg/mL, 46 mg/mL to 46.5 mg/mL, 46.5 mg/mL to 47 mg/mL, 47 mg/mL to 47.5 mg/mL, 47.5 mg/mL to 48 mg/mL, 48 mg/mL to 48.5 mg/mL, 48.5 mg/mL to 49 mg/mL, 49 mg/mL to 49.5 mg/mL and 49.5 mg/mL to 50 mg/mL.

In some embodiments, concentration of Desidustat or pharmaceutically acceptable salts thereof in parenteral pharmaceutical composition is in range of 15 mg/mL to 35 mg/mL. In some embodiments, concentration of Desidustat or pharmaceutically acceptable salts thereof in parenteral pharmaceutical composition is in range of 30 mg/mL to 50 mg/mL. In some embodiments, concentration of Desidustat or pharmaceutically acceptable salts thereof in parenteral pharmaceutical composition is in range of 40 mg/mL to 60 mg/mL. In some embodiments, concentration of Desidustat or pharmaceutically acceptable salts thereof in parenteral pharmaceutical composition is in range of 90 mg/mL to 110 mg/mL.

In some embodiments, concentration of Desidustat or pharmaceutically acceptable salts thereof in parenteral pharmaceutical composition is 25 mg/mL. In some embodiments, concentration of Desidustat or pharmaceutically acceptable salts thereof in parenteral pharmaceutical composition is 40 mg/mL. In some embodiments, concentration of Desidustat or pharmaceutically acceptable salts thereof in parenteral pharmaceutical composition is 50 mg/mL. In some embodiments, concentration of Desidustat or pharmaceutically acceptable salts thereof in parenteral pharmaceutical composition is 100 mg/mL.

Suitable pharmaceutically acceptable excipients selected from but are not limited to an isotonic agent/cosolvent and an alkalizing agent.

An isotonic agent/cosolvent selected from but are not limited to Albumin, Dimethylacetamide, Glycerine, Propylene Glycol, Polyethylene Glycol, PEG 200, PEG 300, PEG 400, Sodium Chloride, Mannitol, Trehalose, Dextrose, Hydroxypropyl Betadex, Potassium Chloride, Sulfobutylether O-Cyclodextrin, and Butylene Glycol or suitable mixture thereof.

In an aspect, the present invention relates to a parenteral composition of Desidustat or pharmaceutically acceptable salts thereof wherein an amount of isotonic agent/cosolvent used is in the range of 0.001% to 20% w/v.

Further, amount of isotonic agent/cosolvent in pharmaceutical composition used is in the range of 0.001% to 0.005%, 0.005% to 0.01%, 0.01% to 0.02%, 0.02% to 0.03%, 0.03% to 0.04%, 0.04% to 0.05%, 0.05% to 0.06%, 0.06% to 0.07%, 0.07% to 0.08%, 0.08% to 0.09%, 0.09% to 0.1%, 0.1% to 0.5%, 0.5% to 1%, 1% to 1.5%, 1.5% to 2%, 2% to 2.5%, 2.5% to 3%, 3% to 3.5%, 3.5% to 4%, 4% to 4.5%, 4.5% to 5%, 5% to 5.5%, 5.5% to 6%, 6% to 6.5%, 6.5% to 7%, 7% to 7.5%, 7.5% to 8%, 8% to 8.5%, 8.5% to 9%, 9% to 9.5%, 9.5% to 10%, 10% to 10.5%, 10.5% to 11%, 11% to 11.5%, 11.5% to 12%, 12% to 12.5%, 12.5% to 13%, 13% to 13.5%, 13.5% to 14%, 14% to 14.5%, 14.5% to 15%, 15% to 15.5%, 15.5% to 16%, 16% to 16.5%, 16.5% to 17%, 17% to 17.5%, 17.5% to 18%, 18% to 18.5%, 18.5% to 19%, 19% to 19.5% and 19.5% to 20%.

In certain embodiments, the amount of isotonic agent/cosolvent in pharmaceutical composition used is in the range of 0.01% to 0.05%, 0.05% to 0.1%, 0.1% to 0.15%, 0.15% to 0.2%, 0.2% to 0.25%, 0.25% to 0.3%, 0.3% to 0.35%, 0.35% to 0.4%, 0.4% to 0.45%, 0.45% to 0.5%, 0.5% to 0.55%, 0.55% to 0.6%, 0.6% to 0.65%, 0.65% to 0.7%, 0.7% to 0.75%, 0.75% to 0.8%, 0.8% to 0.85%, 0.85% to 0.9%, 0.9% to 0.95%, 0.95% to 1%, 1% to 1.05%, 1.05% to 1.1%, 1.1% to 1.15%, 1.15% to 1.2%, 1.2% to 1.25%, 1.25% to 1.3%, 1.3% to 1.35%, 1.35% to 1.4%, 1.4% to 1.45%, 1.45% to 1.5%, 1.5% to 1.55%, 1.55% to 1.6%, 1.6% to 1.65%, 1.65% to 1.7%, 1.7% to 1.75%, 1.75% to 1.8%, 1.8% to 1.85%, 1.85% to 1.9%, 1.9% to 1.95%, 1.95% to 2%, 2% to 2.15%, 2.15% to 2.2%, 2.2% to 2.25%, 2.25% to 2.3%, 2.3% to 2.35%, 2.35% to 2.4%, 2.4% to 2.45%, 2.45% to 2.5%, 2.5% to 2.55%, 2.55% to 2.6%, 2.6% to 2.65%, 2.65% to 2.7%, 2.7% to 2.75%, 2.75% to 2.8%, 2.8% to 2.85%, 2.85% to 2.9%, 2.9% to 2.95%, 2.95% to 3%, 3% to 3.05%, 3.05% to 3.1%, 3.1% to 3.15%, 3.15% to 3.2%, 3.2% to 3.25%, 3.25% to 3.3%, 3.3% to 3.35%, 3.35% to 3.4%, 3.4% to 3.45%, 3.45% to 3.5%, 3.5% to 3.55%, 3.55% to 3.6%, 3.6% to 3.65%, 3.65% to 3.7%, 3.7% to 3.75%, 3.75% to 3.8%, 3.8% to 3.85%, 3.85% to 3.9%, 3.9% to 3.95%, 3.95% to 4%, 4% to 4.05%, 4.05% to 4.1%, 4.1% to 4.15%, 4.15% to 4.2%, 4.2% to 4.25%, 4.25% to 4.3%, 4.3% to 4.35%, 4.35% to 4.4%, 4.4% to 4.45%, 4.45% to 4.5%, 4.5% to 4.55%, 4.55% to 4.6%, 4.6% to 4.65%, 4.65% to 4.7%, 4.7% to 4.75%, 4.75% to 4.8%, 4.8% to 4.85%, 4.85% to 4.9%, 4.9% to 4.95% and 4.95% to 5%.

In certain embodiments, the amount of isotonic agent/cosolvent in pharmaceutical composition used is in the range of 0.1% to 1.5%.

An alkalizing agent may be selected from but is not limited to Triethanolamine (Trolamine), Calcium Hydroxide, Diethanolamine, Ethanolamine (Monoethanolamine), Potassium Hydroxide, Sodium Carbonate, Sodium Citrate, Sodium Hydroxide, Meglumine, Sodium lactate, Tromethamine or suitable mixture thereof.

In an aspect, the present invention relates to a parenteral composition of Desidustat or pharmaceutically acceptable salts thereof wherein an amount of alkalizing agent used is in the range of 0.001% to 20% w/v.

Further, amount of alkalizing agent in pharmaceutical composition used is in the range of 0.001% to 0.005%, 0.005% to 0.01%, 0.01% to 0.02%, 0.02% to 0.03%, 0.03% to 0.04%, 0.04% to 0.05%, 0.05% to 0.06%, 0.06% to 0.07%, 0.07% to 0.08%, 0.08% to 0.09%, 0.09% to 0.1%, 0.1% to 0.5%, 0.5% to 1%, 1% to 1.5%, 1.5% to 2%, 2% to 2.5%, 2.5% to 3%, 3% to 3.5%, 3.5% to 4%, 4% to 4.5%, 4.5% to 5%, 5% to 5.5%, 5.5% to 6%, 6% to 6.5%, 6.5% to 7%, 7% to 7.5%, 7.5% to 8%, 8% to 8.5%, 8.5% to 9%, 9% to 9.5%, 9.5% to 10%, 10% to 10.5%, 10.5% to 11%, 11% to 11.5%, 11.5% to 12%, 12% to 12.5%, 12.5% to 13%, 13% to 13.5%, 13.5% to 14%, 14% to 14.5%, 14.5% to 15%, 15% to 15.5%, 15.5% to 16%, 16% to 16.5%, 16.5% to 17%, 17% to 17.5%, 17.5% to 18%, 18% to 18.5%, 18.5% to 19%, 19% to 19.5% and 19.5% to 20%.

In certain embodiments, the amount of alkalizing agent used in the pharmaceutical composition is in the range of 0.01% to 0.05%, 0.05% to 0.1%, 0.1% to 0.15%, 0.15% to 0.2%, 0.2% to 0.25%, 0.25% to 0.3%, 0.3% to 0.35%, 0.35% to 0.4%, 0.4% to 0.45%, 0.45% to 0.5%, 0.5% to 0.55%, 0.55% to 0.6%, 0.6% to 0.65%, 0.65% to 0.7%, 0.7% to 0.75%, 0.75% to 0.8%, 0.8% to 0.85%, 0.85% to 0.9%, 0.9% to 0.95%, 0.95% to 1%, 1% to 1.05%, 1.05% to 1.1%, 1.1% to 1.15%, 1.15% to 1.2%, 1.2% to 1.25%, 1.25% to 1.3%, 1.3% to 1.35%, 1.35% to 1.4%, 1.4% to 1.45%, 1.45% to 1.5%, 1.5% to 1.55%, 1.55% to 1.6%, 1.6% to 1.65%, 1.65% to 1.7%, 1.7% to 1.75%, 1.75% to 1.8%, 1.8% to 1.85%, 1.85% to 1.9%, 1.9% to 1.95%, 1.95% to 2%, 2% to 2.15%, 2.15% to 2.2%, 2.2% to 2.25%, 2.25% to 2.3%, 2.3% to 2.35%, 2.35% to 2.4%, 2.4% to 2.45%, 2.45% to 2.5%, 2.5% to 2.55%, 2.55% to 2.6%, 2.6% to 2.65%, 2.65% to 2.7%, 2.7% to 2.75%, 2.75% to 2.8%, 2.8% to 2.85%, 2.85% to 2.9%, 2.9% to 2.95%, 2.95% to 3%, 3% to 3.05%, 3.05% to 3.1%, 3.1% to 3.15%, 3.15% to 3.2%, 3.2% to 3.25%, 3.25% to 3.3%, 3.3% to 3.35%, 3.35% to 3.4%, 3.4% to 3.45%, 3.45% to 3.5%, 3.5% to 3.55%, 3.55% to 3.6%, 3.6% to 3.65%, 3.65% to 3.7%, 3.7% to 3.75%, 3.75% to 3.8%, 3.8% to 3.85%, 3.85% to 3.9%, 3.9% to 3.95%, 3.95% to 4%, 4% to 4.05%, 4.05% to 4.1%, 4.1% to 4.15%, 4.15% to 4.2%, 4.2% to 4.25%, 4.25% to 4.3%, 4.3% to 4.35%, 4.35% to 4.4%, 4.4% to 4.45%, 4.45% to 4.5%, 4.5% to 4.55%, 4.55% to 4.6%, 4.6% to 4.65%, 4.65% to 4.7%, 4.7% to 4.75%, 4.75% to 4.8%, 4.8% to 4.85%, 4.85% to 4.9%, 4.9% to 4.95% and 4.95% to 5%.

In certain embodiments, the amount of alkalizing agent in pharmaceutical composition used is in the range of 0.5% to 3%.

In an embodiment, parenteral pharmaceutical composition may use as Intramuscular, Subcutaneous, Intravenous. Parenteral solution may use as ready to use solution. Parenteral solution is in the form of injectable.

In certain embodiments, parenteral pharmaceutical composition of Desidustat or pharmaceutically acceptable salts thereof can be further characterized according to the dose of compound administered to a subject, where subject is animal or human.

In another embodiment, the parenteral pharmaceutical composition is administered to the subject once a day, twice a day or thrice a day or on alternate day for at least 2 weeks.

### Use of Oral Composition of Desidustat or Pharmaceutically Acceptable Salts Thereof for the Treatment of Radiation Induced Toxicity

In an embodiment, present invention provides a use of oral composition of Desidustat or pharmaceutically acceptable salts thereof optionally with one or more pharmaceutically acceptable excipients for the treatment of radiation induced toxicity.

In an embodiment, radiation induced toxicity includes acute radiation syndrome.

In another embodiment, Acute Radiation Syndrome is selected from Hematopoietic Acute Radiation Syndrome (H-ARS), Gastrointestinal Syndrome and Cardiovascular/Central Nervous System Syndrome.

Hematopoietic Acute Radiation Syndrome occurs when the whole body or a significant portion of body is exposed to a high dose of penetrating ionizing radiation, typically between 0.7 and 10 Gray (Gy), delivered over a short period (minutes to hours).

An oral composition of Desidustat is useful for the treatment of Hematopoietic Acute Radiation Syndrome.

The suitable pharmaceutically acceptable salts of Desidustat may be selected from suitable inorganic metal salt or an organic amine salt.

In certain embodiments, the inorganic metal salts can be selected from calcium salt, sodium salt, potassium salt, lithium salt, barium salt, strontium salt, magnesium salt, cesium salt, copper salt, cobalt salt, iron salt, manganese salt, lead salt, aluminium salt, cadmium salt, silver salt, and zinc salt, and the like.

In certain embodiments, the organic amine salts, also referred to herein as organic ammonium salts can be selected from methylammonium salt, dimethylammonium salt, ethylammonium salt, diethyl ammonium salt, n-propyl ammonium salt, isopropyl ammonium salt, diisopropyl ammonium salt, N-methyl isopropyl ammonium salt, n-butyl ammonium salt, t-butyl ammonium salt, 2-butammonium salt, 1,2-ethane diammonium salt, N-methylglucammonium salt, N,N,N-trimethyl ethanolammonium hydroxide (choline) salt, tromethammmonium salt, cyclohexylammonium salt, N-methyl cyclohexylammonium salt, guanidinium salt, N-(4-aminobutyl) guanidinium salt, dicyclohexylammonium salt, benzene-methanammonium salt, ethanolammonium salt, diethanolammonium salt, tris-(hydroxymethyl)methylammonium salt, hydroxylammonium salt, methanammonium salt, benzylammonium salt, N-methylbenzylammonium salt, N-ethyl benzylammonium salt, 4-methoxybenzylammonium salt, pyrrolidinium salt, piperidinium salt, piperazinium salt, morpholinium salt, 2-aminopyrimidinium salt, alaninium salt, lysinium salt, argininium salt, histidinium salt, threoninium salt, prolinium salt, glutammonium salt, glycinium salt, 2-thiopheneethanammonium salt, (2S)-3,3-dimethyl-2-butanammonium salt, cyclopentanammonium salt, cycloheptanammonium salt, meglummonium salt, bethammonium salt, dibenzylammonium salt, diphenylammonium salt, α-naphthylammonium salt, O-phenylenediammonium salt, 1,3-Diaminopropammonium salt, (S)-α-naphthylethylammonium salt, (S)-3-methoxyphenylethylammonium salt, (S)-4-methoxyphenylethylammonium salt, (S)-4-chlorophenylethylammonium salt, (S)-4-methylphenylethylammonium salt, cinchoninium salt, cinchonidinium salt, (−)-quininium salt, triethanolammonium salt, imidazolium salt, ethylenediammonium salt, polammonium salt, morpholinium 4-(2-hydroxyethyl) salt, N—N-diethylethanolammonium salt, deammonium salt, hydrabammonium salt, betammonium salt, adamantanammonium salt, L-adamantanmethylammonium salt, tritylammonium salt, glucammonium salt, N-methyl pyrrolidinium salt, ureammonium salt, procammonium salt, metformmonium salt, hexane1-6-diammonium salt, 2-(2-aminoethoxy)ethanammonium salt, N-methylmorpholinium salt, and N-ethylmorpholinium salt.

In an another embodiment, the uncoated tablet comprises concentration of Desidustat in the range of 1% w/w to 90% w/w, dose of Desidustat in the range of 1% w/w to 80% w/w, dose of Desidustat in the range of 1% w/w to 70% w/w, dose of Desidustat in the range of 1% w/w to 60% w/w, dose of Desidustat in the range of 1% w/w to 50% w/w, dose of Desidustat in the range of 1% w/w to 40% w/w, dose of Desidustat in the range of 1% w/w to 30% w/w, dose of Desidustat in the range of 1% w/w to 20% w/w or dose of Desidustat in the range of 1% w/w to 10% w/w.

In one embodiment, the present invention provides an uncoated tablet comprises Desidustat or pharmaceutically acceptable salts thereof and one or more a pharmaceutically acceptable excipient.

In an embodiment pharmaceutical excipient according to present invention can be selected from diluents or fillers, disintegrants, binder, lubricants, glidants, film forming agents, plasticizers, opacifier, solvents, and the like as known in the art.

As used herein the term “diluent or filler” refers to substances that are used to dilute the active pharmaceutical ingredient prior to delivery. Diluents can also serve as stabilizers.

Non-limiting examples of diluents include starch and its processed and co-processed derivatives, saccharides, di saccharides, sucrose, lactose, polysaccharides, cellulose, cellulose ethers, cellulose acetate, hydroxypropyl cellulose, sugar alcohols, xylitol, sorbitol, maltitol, lactitol, microcrystalline cellulose, magnesium or calcium or sodium carbonate, lactose, lactose monohydrate, di-calcium phosphate, compressible sugars, di basic calcium phosphate dihydrate, mannitol lactose anhydrous, magnesium oxide, maltodextrin, maltose, pullulan, sodium alginate, sodium bicarbonate, calcium silicate, calcium sulphate, cell and tribasic calcium phosphate or suitable combinations thereof.

As used herein the term “disintegrant or disintegrating agents” refers to a substance which, upon addition to a solid preparation, facilitates its break-up or disintegration after administration and permits the release of an active ingredient as efficiently as possible to allow for its rapid dissolution. Non-limiting examples of disintegrants include maize starch, sodium starch glycolate, croscarmellose sodium, crospovidone, microcrystalline cellulose, modified corn starch, sodium carboxymethyl starch, povidone, pregelatinized starch, agar, carboxymethyl cellulose calcium or sodium, colloidal silicon dioxide, chitosan, docusate sodium, hydroxyl propyl cellulose, magnesium aluminium silicate, maltose, methyl cellulose, polacrilin potassium, and alginic acid or suitable combinations thereof. As used herein the term “binder” refers to any pharmaceutically acceptable substance which can be used to bind the active and inert components together to maintain cohesive and discrete portions. Non-limiting examples of binders chitosan, hydrogenated castor oil, sodium alginate, carbomers, cellulose acetate phthalate, povidone, sugar, hydroxypropylmethyl-cellulose, hydroxypropylcellulose, starch, alginic acid, pregelatinized starch, acacia, tragakanth, ethylcellulose, acrylic and methacrylic acid co polymers or suitable combinations thereof.

As used herein the term “lubricant” refers to an excipient, which is added to a powder blend to prevent the compacted powder mass from sticking to the equipment during the tableting or encapsulation process. It aids the ejection of the tablet form the dies, and can improve powder flow. Non-limiting examples of lubricants include magnesium stearate, stearic acid, silica, fats, zinc or sucrose or sodium or calcium stearate, castor oil, hydrogenated castor oil, polyethylene glycol and its derivatives, sodium stearyl fumarate, talc, or fatty acids including lauric acid, oleic acid, glyceryl behenate, glyceryl monostearate, and C₁-C₁₀ fatty acid or suitable combinations thereof.

As used herein the term “glidant” is intended to mean agents used in tablet and capsule formulations to improve flow-properties during tablet compression and to produce an anti caking effect. Non-limiting examples of glidants include colloidal silicon dioxide, talc, fumed silica, starch, starch derivatives, and bentonite or suitable combinations thereof.

As used herein the term “film forming agents” refers to but not limited to hydroxypropyl methylcellulose, methylcellulose, ethylcellulose, hydroxypropyl cellulose, povidone, polydextrose, lactose, maltodextrin, acrylic polymer or suitable combinations thereof.

As used herein the term “plasticizer” refers to but not limited to polyols like polyethylene glycols, propylene glycol, glycerol (glycerine), organic esters like phthalate esters (diethyl, dibutyl), dibutyl sebacete, citrate esters (triethyl, acetyl triethyl, acetyl tributyl), triacetin, Oils/glycerides like castor oil; acetylated monoglycerides, fractionated coconut oil or suitable combinations thereof.

As used herein the term “opacifier” refers to but not limited to titanium dioxide, talc, sunset yellow, tartrazine, erythrosine, iron oxide yellow, red and black, carmine, anthocyanins. allura Red AC, allura Red AC aluminum lake, indigotine, indigotine aluminum lake or suitable combinations thereof.

One or more “solvents” used in the formulation are selected from water, acetone, chloroform, dichlorome thane, ethyl alcohol, ethyl acetate, methyl alcohol, isopropyl alcohol, N,N-dimethyl formamide and combinations thereof and other such materials known to those of ordinary skill in the art.

In an another embodiment, the uncoated tablet comprises from about 1% to about 90% w/w Desidustat, microcrystalline cellulose from about 2% w/w to about 90% w/w, croscarmellose sodium from about 0.5% w/w to 10% w/w, lactose monohydrate from about 2% w/w to about 90% w/w, hypromellose 3 cps from about 0.5% w/w to about 10% w/w, Talc from about 0.5% w/w to about 3% w/w, magnesium Stearate from about 0.5% w/w to about 5% w/w of the total composition, polyvinyl pyrrolidone from about 0.5% w/w to about 10% w/w, starch from about 1% w/w to about 20% w/w based on the weight of uncoated tablet.

In certain embodiments, Desidustat or pharmaceutically acceptable salts thereof can be further characterized according to the dose of compound administered to a subject, where subject is animal or human.

In another embodiment, the oral pharmaceutical composition is administered to the subject once a day, twice a day or thrice a day or on alternate day for at least 2 weeks.

The use of Desidustat or a pharmaceutically acceptable salt thereof to the subject increases hemoglobin and/or red blood cell (RBC) count relative to baseline of a subject due to the therapeutic method (e.g., administering Desidustat or a pharmaceutically acceptable salt thereof to the subject). For example, in certain embodiments, the use Desidustat or a pharmaceutically acceptable salt thereof increases hemoglobin and/or red blood cell (RBC) count relative to baseline of the subject. In certain embodiments, the use of Desidustat or a pharmaceutically acceptable salt thereof increases hemoglobin and/or red blood cell (RBC) count relative to baseline of the subject by at least 5%. In certain embodiments, the use of Desidustat or a pharmaceutically acceptable salt thereof increases hemoglobin and/or red blood cell (RBC) count relative to baseline of the subject by at least 10%. In certain embodiments, the use of Desidustat or a pharmaceutically acceptable salt thereof increases hemoglobin and/or red blood cell (RBC) count relative to baseline of the subject by at least 15%. In certain embodiments, the use of Desidustat or a pharmaceutically acceptable salt thereof increases hemoglobin and/or red blood cell (RBC) count relative to baseline of the subject by at least 20%. In certain embodiments, the use of Desidustat or a pharmaceutically acceptable salt thereof increases hemoglobin and/or red blood cell (RBC) count relative to baseline of the subject by at least 25%. In certain embodiments, the use of Desidustat or a pharmaceutically acceptable salt thereof increases hemoglobin and/or red blood cell (RBC) count relative to baseline of the subject by at least 30%. In certain embodiments, the use of Desidustat or a pharmaceutically acceptable salt thereof increases hemoglobin and/or red blood cell (RBC) count relative to baseline of the subject by at least 35%. In certain embodiments, the use of Desidustat or a pharmaceutically acceptable salt thereof increases hemoglobin and/or red blood cell (RBC) count relative to baseline of the subject by at least 40%. In certain embodiments, the use of Desidustat or a pharmaceutically acceptable salt thereof increases hemoglobin and/or red blood cell (RBC) count relative to baseline of the subject by at least 45%. In certain embodiments, the use of Desidustat or a pharmaceutically acceptable salt thereof increases hemoglobin and/or red blood cell (RBC) count relative to baseline of the subject by at least 50%.

Desidustat or Pharmaceutically Acceptable Salts Thereof in Combination with Other Suitable Therapeutic Agent for the Treatment of Radiation Induced Toxicity

In an embodiment the present invention provides the administration of Desidustat or pharmaceutically acceptable salts thereof in combination with other suitable agents as therapeutic agent for the treatment of radiation induced toxicity.

In an embodiment, radiation induced toxicity include acute radiation syndrome.

In another embodiment, Acute Radiation Syndrome is selected from Hematopoietic Acute Radiation Syndrome (H-ARS).

Hematopoietic Acute Radiation Syndrome occurs when the whole body or a significant portion of body is exposed to a high dose of penetrating ionizing radiation, typically between 0.7 and 10 Gray (Gy), delivered over a short period (minutes to hours).

Desidustat in combination with other suitable therapeutic agent is useful for the treatment of Hematopoietic Acute Radiation Syndrome.

In an embodiment, concentration of Desidustat or pharmaceutically acceptable salts thereof used in the range disclosed above anywhere in the specification.

In an embodiment, the additional therapeutic agent used is selected from Neupogen (filgrastim), Neulasta (Pegfilgrasim), Leukine (Sargramostim), Nplate (romiplostim).

In certain embodiments, combination can be further characterized according to the dose of compound administered to a subject, where subject is animal or human.

### Parenteral Pharmaceutical Compositions of Roxadustat or its Suitable Pharmaceutically Acceptable Salts

In another embodiment, the present invention provides a parenteral pharmaceutical composition comprises Roxadustat or its pharmaceutically acceptable salt, suitable an alkalizing agent and optionally suitable pharmaceutically acceptable excipients.

In certain embodiments, Roxadustat or its suitable pharmaceutically acceptable salts, for administration to a subject at a concentration in the range of 1 mg/mL to 250 mg/mL.

Suitable pharmaceutically acceptable excipients selected from but are not limited to an isotonic agent/cosolvent and an alkalizing agent.

In an aspect, the present invention relates to a parenteral composition of Roxadustat or its suitable pharmaceutically acceptable salts wherein an amount of isotonic agent/cosolvent and an alkalizing agent used is in the range of 0.001% to 20% w/v.

In an embodiment, parenteral composition may be administered as Intramuscular, Subcutaneous, Intravenous. Parenteral solution may be use as ready to use solution.

### Modes of Administration

The compositions of the present invention can be delivered directly or in pharmaceutical compositions containing excipients, as is well known in the art. The present methods of treatment involve administration of an effective amount of a compound of the present invention to a subject having or at risk for having radiation induced toxicity.

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

The present disclosure includes the following enumerated embodiments:
1. A parenteral pharmaceutical composition comprises Desidustat or a pharmaceutically acceptable salt thereof.
2. The parenteral pharmaceutical composition of embodiment 1, wherein the parenteral pharmaceutical composition comprises a suitable pharmaceutically acceptable excipient selected from a suitable isotonic agent/cosolvent and a suitable alkalizing agent.
3. The parenteral pharmaceutical composition of embodiment 1 or 2, wherein the pharmaceutically acceptable salt of Desidustat is a suitable inorganic metal salt or an organic amine salt.
4. The parenteral pharmaceutical composition of embodiment 3, wherein the inorganic metal salt is selected from calcium salt, sodium salt, potassium salt, lithium salt, barium salt, strontium salt, magnesium salt, cesium salt, copper salt, cobalt salt, iron salt, manganese salt, lead salt, aluminium salt, cadmium salt, silver salt, and zinc salt; and the organic amine salt is selected from methylammonium salt, dimethylammonium salt, ethylammonium salt, diethyl ammonium salt, n-propyl ammonium salt, isopropyl ammonium salt, diisopropyl ammonium salt, N-methyl isopropyl ammonium salt, n-butyl ammonium salt, t-butyl ammonium salt, 2-butammonium salt, 1,2-ethane diammonium salt, N-methylglucammonium salt, N,N,N-trimethyl ethanolammonium hydroxide (choline) salt, tromethammmonium salt, cyclohexylammonium salt, N-methyl cyclohexylammonium salt, guanidinium salt, N-(4-aminobutyl) guanidinium salt, dicyclohexylammonium salt, benzene-methanammonium salt, ethanolammonium salt, diethanolammonium salt, tris-(hydroxymethyl)methylammonium salt, hydroxylammonium salt, methanammonium salt, benzylammonium salt, N-methylbenzylammonium salt, N-ethyl benzylammonium salt, 4-methoxybenzylammonium salt, pyrrolidinium salt, piperidinium salt, piperazinium salt, morpholinium salt, 2-aminopyrimidinium salt, alaninium salt, lysinium salt, argininium salt, histidinium salt, threoninium salt, prolinium salt, glutammonium salt, glycinium salt, 2-thiopheneethanammonium salt, (2S)-3,3-dimethyl-2-butanammonium salt, cyclopentanammonium salt, cycloheptanammonium salt, meglummonium salt, bethammonium salt, dibenzylammonium salt, diphenylammonium salt, α-naphthylammonium salt, O-phenylenediammonium salt, 1,3-Diaminopropammonium salt, (S)-α-naphthylethylammonium salt, (S)-3-methoxyphenylethylammonium salt, (S)-4-methoxyphenylethylammonium salt, (S)-4-chlorophenylethylammonium salt, (S)-4-methylphenylethylammonium salt, cinchoninium salt, cinchonidinium salt, (−)-quininium salt, triethanolammonium salt, imidazolium salt, ethylenediammonium salt, polammonium salt, morpholinium 4-(2-hydroxyethyl) salt, N—N-diethylethanolammonium salt, deammonium salt, hydrabammonium salt, betammonium salt, adamantanammonium salt, L-adamantanmethylammonium salt, tritylammonium salt, glucammonium salt, N-methyl pyrrolidinium salt, ureammonium salt, procammonium salt, metformmonium salt, hexane1-6-diammonium salt, 2-(2-aminoethoxy)ethanammonium salt, N-methylmorpholinium salt, and N-ethylmorpholinium salt.
5. The parenteral pharmaceutical composition of any of embodiments 1-4, wherein the parenteral pharmaceutical composition comprises Desidustat or a pharmaceutically acceptable salt thereof in a concentration selected from 1 mg/mL to 250 mg/mL, 1 mg/mL to 200 mg/mL, 1 mg/mL to 150 mg/mL, 1 mg/mL to 125 mg/mL, 1 mg/mL to 100 mg/mL, 1 mg/mL to 75 mg/mL, 1 mg/mL to 50 mg/mL, 1 mg/mL to 25 mg/mL and 1 mg/mL to 10 mg/mL.
6. The parenteral pharmaceutical composition of embodiment 5, wherein the parenteral pharmaceutical composition comprises Desidustat or a pharmaceutically acceptable salt thereof in a concentration of 1 mg/mL to 125 mg/mL.
7. The parenteral pharmaceutical composition of embodiment 5 and 6, wherein the parenteral pharmaceutical composition comprises the compound in a concentration selected from 25 mg/mL, 40 mg/mL, 50 mg/mL and 100 mg/mL.
8. The parenteral pharmaceutical composition of any of embodiments 2-7, wherein the isotonic agent/cosolvent is selected from Albumin, Dimethylacetamide, Glycerine, Propylene Glycol, Polyethylene Glycol, PEG 200, PEG 300, PEG 400, Sodium Chloride, Mannitol, Trehalose, Dextrose, Hydroxypropyl Betadex, Potassium Chloride, Sulfobutylether O-Cyclodextrin, Butylene Glycol and a suitable mixture thereof; and the alkalizing agent is selected from Triethanolamine (Trolamine), Calcium Hydroxide, Diethanolamine, Ethanolamine (Monoethanolamine), Potassium Hydroxide, Sodium Carbonate, Sodium Citrate, Sodium Hydroxide, Meglumine, Sodium lactate, Tromethamine and a suitable mixture thereof.
9. The parenteral pharmaceutical composition of embodiment 8, wherein the isotonic agent/cosolvent is selected from glycerine, sodium chloride, and a suitable mixture thereof; and the alkalizing agent is selected from sodium hydroxide, meglumine, tromethamine and a suitable mixture thereof.
10. The parenteral pharmaceutical composition of any of embodiments 2-9 wherein % w/v of the isotonic agent/cosolvent is selected from 0.001% to 0.005%, 0.005% to 0.01%, 0.01% to 0.02%, 0.02% to 0.03%, 0.03% to 0.04%, 0.04% to 0.05%, 0.05% to 0.06%, 0.06% to 0.07%, 0.07% to 0.08%, 0.08% to 0.09%, 0.09% to 0.1%, 0.1% to 0.5%, 0.5% to 1%, 1% to 1.5%, 1.5% to 2%, 2% to 2.5%, 2.5% to 3%, 3% to 3.5%, 3.5% to 4%, 4% to 4.5%, 4.5% to 5%, 5% to 5.5%, 5.5% to 6%, 6% to 6.5%, 6.5% to 7%, 7% to 7.5%, 7.5% to 8%, 8% to 8.5%, 8.5% to 9%, 9% to 9.5%, 9.5% to 10%, 10% to 10.5%, 10.5% to 11%, 11% to 11.5%, 11.5% to 12%, 12% to 12.5%, 12.5% to 13%, 13% to 13.5%, 13.5% to 14%, 14% to 14.5%, 14.5% to 15%, 15% to 15.5%, 15.5% to 16%, 16% to 16.5%, 16.5% to 17%, 17% to 17.5%, 17.5% to 18%, 18% to 18.5%, 18.5% to 19%, 19% to 19.5%, and 19.5% to 20%; and % w/v of the alkalizing agent is selected from 0.001% to 0.005%, 0.005% to 0.01%, 0.01% to 0.02%, 0.02% to 0.03%, 0.03% to 0.04%, 0.04% to 0.05%, 0.05% to 0.06%, 0.06% to 0.07%, 0.07% to 0.08%, 0.08% to 0.09%, 0.09% to 0.1%, 0.1% to 0.5%, 0.5% to 1%, 1% to 1.5%, 1.5% to 2%, 2% to 2.5%, 2.5% to 3%, 3% to 3.5%, 3.5% to 4%, 4% to 4.5%, 4.5% to 5%, 5% to 5.5%, 5.5% to 6%, 6% to 6.5%, 6.5% to 7%, 7% to 7.5%, 7.5% to 8%, 8% to 8.5%, 8.5% to 9%, 9% to 9.5%, 9.5% to 10%, 10% to 10.5%, 10.5% to 11%, 11% to 11.5%, 11.5% to 12%, 12% to 12.5%, 12.5% to 13%, 13% to 13.5%, 13.5% to 14%, 14% to 14.5%, 14.5% to 15%, 15% to 15.5%, 15.5% to 16%, 16% to 16.5%, 16.5% to 17%, 17% to 17.5%, 17.5% to 18%, 18% to 18.5%, 18.5% to 19%, 19% to 19.5%, and 19.5% to 20%.
11. The parenteral pharmaceutical composition of any of embodiments 2-10 wherein % w/v of the isotonic agent/cosolvent is selected from 0.1% to 1.5%; and % w/v of the alkalizing agent is selected from 0.5% to 3%.
12. A parenteral pharmaceutical composition as described in any one of Examples 1-8.
13. A method of treating radiation induced toxicity in a subject, comprises administering a therapeutically effective amount of Desidustat or a pharmaceutically acceptable salt thereof.
14. A method of treating radiation induced toxicity in a subject, comprises administering a therapeutically effective amount of the parenteral pharmaceutical composition of any of embodiments 1-12.
15. A method of treating radiation induced toxicity in a subject comprises administering a therapeutically effective amount of an oral pharmaceutical composition comprises Desidustat or a pharmaceutically acceptable salt thereof.
16. The method of any of embodiments 13-15, wherein the radiation induced toxicity is acute radiation syndrome.
17. The method of embodiment 16, wherein the acute radiation syndrome is hematopoietic acute radiation syndrome (H-ARS).
18. The method of any of embodiments 13-17, wherein the pharmaceutical acceptable salt of Desidustat is a suitable inorganic metal salt or an organic amine salt of Desidustat.
19. The method of embodiment 18, wherein the inorganic metal salt of Desidustat is selected from calcium salt, sodium salt, potassium salt, lithium salt, barium salt, strontium salt, magnesium salt, cesium salt, copper salt, cobalt salt, iron salt, manganese salt, lead salt, aluminium salt, cadmium salt, silver salt, and zinc salt; and the organic amine salt of Desidustat is selected from methylammonium salt, dimethylammonium salt, ethylammonium salt, diethyl ammonium salt, n-propyl ammonium salt, isopropyl ammonium salt, diisopropyl ammonium salt, N-methyl isopropyl ammonium salt, n-butyl ammonium salt, t-butyl ammonium salt, 2-butammonium salt, 1,2-ethane diammonium salt, N-methylglucammonium salt, N,N,N-trimethyl ethanolammonium hydroxide (choline) salt, tromethammmonium salt, cyclohexylammonium salt, N-methyl cyclohexylammonium salt, guanidinium salt, N-(4-aminobutyl) guanidinium salt, dicyclohexylammonium salt, benzene-methanammonium salt, ethanolammonium salt, diethanolammonium salt, tris-(hydroxymethyl)methylammonium salt, hydroxylammonium salt, methanammonium salt, benzylammonium salt, N-methylbenzylammonium salt, N-ethyl benzylammonium salt, 4-methoxybenzylammonium salt, pyrrolidinium salt, piperidinium salt, piperazinium salt, morpholinium salt, 2-aminopyrimidinium salt, alaninium salt, lysinium salt, argininium salt, histidinium salt, threoninium salt, prolinium salt, glutammonium salt, glycinium salt, 2-thiopheneethanammonium salt, (2S)-3,3-dimethyl-2-butanammonium salt, cyclopentanammonium salt, cycloheptanammonium salt, meglummonium salt, bethammonium salt, dibenzylammonium salt, diphenylammonium salt, α-naphthylammonium salt, 0-phenylenediammonium salt, 1,3-Diaminopropammonium salt, (S)-α-naphthylethylammonium salt, (S)-3-methoxyphenylethylammonium salt, (S)-4-methoxyphenylethylammonium salt, (S)-4-chlorophenylethylammonium salt, (S)-4-methylphenylethylammonium salt, cinchoninium salt, cinchonidinium salt, (−)-quininium salt, triethanolammonium salt, imidazolium salt, ethylenediammonium salt, polammonium salt, morpholinium 4-(2-hydroxyethyl) salt, N—N-diethylethanolammonium salt, deammonium salt, hydrabammonium salt, betammonium salt, adamantanammonium salt, L-adamantanmethylammonium salt, tritylammonium salt, glucammonium salt, N-methyl pyrrolidinium salt, ureammonium salt, procammonium salt, metformmonium salt, hexane1-6-diammonium salt, 2-(2-aminoethoxy)ethanammonium salt, N-methylmorpholinium salt, and N-ethylmorpholinium salt.
20. The method of any of embodiments 13-19, wherein Desidustat or a pharmaceutically acceptable salt thereof is administered to the subject at a dose in the range of 1 mg to 500 mg, 1 mg to 400 mg, 1 mg to 300 mg, 1 mg to 250 mg, 1 mg to 200 mg, 1 mg to 150 mg, 1 mg to 100 mg, 1 mg to 75 mg, 1 mg to 50 mg, 1 mg to 25 mg, 100 mg, 50 mg or 25 mg.
21. The method of embodiment 20, wherein the dose of Desidustat or a pharmaceutically acceptable salt thereof is administered to the subject at a dose in the range of 1 to 125 mg.
22. The method of any of embodiments 13-21, wherein the treating increases hemoglobin and/or red blood cell (RBC) count relative to baseline of a subject by at least 5%, 10%, 15%, 20%, 25%, 30%, 35%, 40%, 40%, 45% and 50%.
23. The method any of embodiments 13-21, wherein the concentration of Desidustat or a pharmaceutically acceptable salt thereof in parenteral pharmaceutical composition is selected from 1 mg/mL to 250 mg/mL, 1 mg/mL to 200 mg/mL, 1 mg/mL to 150 mg/mL, 1 mg/mL to 125 mg/mL, 1 mg/mL to 100 mg/mL, 1 mg/mL to 75 mg/mL, 1 mg/mL to 50 mg/mL, 1 mg/mL to 25 mg/mL, and 1 mg/mL to 10 mg/mL and the concentration of Desidustat or a pharmaceutically acceptable salt thereof in parenteral pharmaceutical composition is selected from 1% w/w to 90% w/w, 1% w/w to 80% w/w, 1% w/w to 70% w/w, 1% w/w to 60% w/w, 1% w/w to 50% w/w, 1% w/w to 40% w/w, 1% w/w to 30% w/w, 1% w/w to 20% w/w or 1% w/w to 10% w/w.
24. The method any of embodiments 15-23, wherein the oral composition comprises solubilizers, diluents or fillers, disintegrants, binder, lubricants, glidants, film forming agents, plasticizers, opacifier, solvents, or a mixture thereof.
25. The method of embodiment 24, wherein the diluent or filler is selected from starch and its processed and co-processed derivatives, saccharides, di saccharides, sucrose, lactose, polysaccharides, cellulose, cellulose ethers, cellulose acetate, hydroxypropyl cellulose, sugar alcohols, xylitol, sorbitol, maltitol, lactitol, microcrystalline cellulose, magnesium or calcium or sodium carbonate, lactose, lactose monohydrate, di-calcium phosphate, compressible sugars, di-basic calcium phosphate dihydrate, mannitol lactose anyhydrous, magnesium oxide, maltodextrin, maltose, pullulan, sodium alginate, sodium bicarbonate, calcium silicate, calcium sulphate, cell and tribasic calcium phosphate or suitable combinations thereof, the disintegrant is selected from maize starch, sodium starch glycolate, croscarmellose sodium, crospovidone, microcrystalline cellulose, modified corn starch, sodium carboxymethyl starch, povidone, pregelatinized starch, agar, carboxymethyl cellulose calcium or sodium, colloidal silicon dioxide, chitosan, docusate sodium, hydroxyl propyl cellulose, magnesium aluminium silicate, maltose, methyl cellulose, polacrilin potassium, and alginic acid or suitable combinations thereof, the binder is selected from chitosan, hydrogenated castor oil, sodium alginate, carbomers, cellulose acetate phthalate, povidone, sugar, hydroxypropylmethyl-cellulose, hydroxypropylcellulose, starch, alginic acid, pregelatinized starch, acacia, tragakanth, ethylcellulose, acrylic and methacrylic acid co polymers or suitable combinations thereof, the lubricant is selected from magnesium stearate, stearic acid, silica, fats, zinc or sucrose or sodium or calcium stearate, castor oil, hydrogenated castor oil. Polyethylene glycol and its derivatives, sodium stearyl fumarate, talc, or fatty acids including lauric acid, oleic acid, glyceryl behenate, glyceryl monostearate, and C/CIO fatty acid or suitable combinations thereof, the glidant is selected from colloidal silicon dioxide, talc, fumed silica, starch, starch derivatives, and bentonite or suitable combinations thereof, film forming agent is selected from hydroxypropyl methylcellulose, methylcellulose, ethylcellulose, hydroxypropyl cellulose, povidone, polydextrose, lactose, maltodextrin, acrylic polymer or suitable combinations thereof, the plasticizers is selected from polyols like polyethylene glycols PEG, propylene glycol, glycerol (glycerine), organic esters like phthalate esters (diethyl, dibutyl), dibutyl sebacete, citrate esters (triethyl, acetyl triethyl, acetyl tributyl), triacetin, Oils/glycerides like castor oil; acetylated monoglycerides, fractionated coconut oil or suitable combinations thereof, the opacifier is selected from titanium dioxide, talc, sunset yellow, Tartrazine, Erythrosine, iron oxide yellow, red and black, Carmine, Anthocyanins. Allura Red AC, Allura Red AC aluminum lake, Indigotine, Indigotine aluminum lake or suitable combinations thereof; solvents is selected from water, acetone, chloroform, dichlorome thane, ethyl alcohol, ethyl acetate, methyl alcohol, isopropyl alcohol, N,N-dimethyl formamide and combinations thereof.
26. Use of Desidustat or a pharmaceutically acceptable salt thereof, for the treatment of radiation induced toxicity in a subject in need thereof.
27. Use of the parenteral pharmaceutical composition of any of embodiments 1-12, for the treatment of radiation induced toxicity in a subject in need thereof.
28. Use of an oral pharmaceutical composition of Desidustat or a pharmaceutically acceptable salt thereof, for the treatment of radiation induced toxicity in a subject in need thereof.
29. The use of any of embodiments 26-28, wherein radiation induced toxicity is acute radiation syndrome.
30. The use of embodiment 29, wherein Acute Radiation Syndrome is selected from hematopoietic acute radiation syndrome (H-ARS).
31. The use of any of embodiments 26-30, wherein the pharmaceutical acceptable salt of Desidustat is selected from suitable an inorganic metal salt or an organic amine salt.
32. The use of embodiment 31, wherein the inorganic metal salt of Desidustat is selected from calcium salt, sodium salt, potassium salt, lithium salt, barium salt, strontium salt, magnesium salt, cesium salt, copper salt, cobalt salt, iron salt, manganese salt, lead salt, aluminium salt, cadmium salt, silver salt, and zinc salt; and the organic amine salt of Desidustat is selected from methylammonium salt, dimethylammonium salt, ethylammonium salt, diethyl ammonium salt, n-propyl ammonium salt, isopropyl ammonium salt, diisopropyl ammonium salt, N-methyl isopropyl ammonium salt, n-butyl ammonium salt, t-butyl ammonium salt, 2-butammonium salt, 1,2-ethane diammonium salt, N-methylglucammonium salt, N,N,N-trimethyl ethanolammonium hydroxide (choline) salt, tromethammmonium salt, cyclohexylammonium salt, N-methyl cyclohexylammonium salt, guanidinium salt, N-(4-aminobutyl) guanidinium salt, dicyclohexylammonium salt, benzene-methanammonium salt, ethanolammonium salt, diethanolammonium salt, tris-(hydroxymethyl)methylammonium salt, hydroxylammonium salt, methanammonium salt, benzylammonium salt, N-methylbenzylammonium salt, N-ethyl benzylammonium salt, 4-methoxybenzylammonium salt, pyrrolidinium salt, piperidinium salt, piperazinium salt, morpholinium salt, 2-aminopyrimidinium salt, alaninium salt, lysinium salt, argininium salt, histidinium salt, threoninium salt, prolinium salt, glutammonium salt, glycinium salt, 2-thiopheneethanammonium salt, (2S)-3,3-dimethyl-2-butanammonium salt, cyclopentanammonium salt, cycloheptanammonium salt, meglummonium salt, bethammonium salt, dibenzylammonium salt, diphenylammonium salt, α-naphthylammonium salt, O-phenylenediammonium salt, 1,3-Diaminopropammonium salt, (S)-α-naphthylethylammonium salt, (S)-3-methoxyphenylethylammonium salt, (S)-4-methoxyphenylethylammonium salt, (S)-4-chlorophenylethylammonium salt, (S)-4-methylphenylethylammonium salt, cinchoninium salt, cinchonidinium salt, (−)-quininium salt, triethanolammonium salt, imidazolium salt, ethylenediammonium salt, polammonium salt, morpholinium 4-(2-hydroxyethyl) salt, N—N-diethylethanolammonium salt, deammonium salt, hydrabammonium salt, betammonium salt, adamantanammonium salt, L-adamantanmethylammonium salt, tritylammonium salt, glucammonium salt, N-methyl pyrrolidinium salt, ureammonium salt, procammonium salt, metformmonium salt, hexane1-6-diammonium salt, 2-(2-aminoethoxy)ethanammonium salt, N-methylmorpholinium salt, and N-ethylmorpholinium salt.
33. The use of any of embodiments 26-32, wherein Desidustat or a pharmaceutically acceptable salt thereof is administered in a dose selected from 1 mg to 500 mg, 1 mg to 400 mg, 1 mg to 300 mg, 1 mg to 250 mg, 1 mg to 200 mg, 1 mg to 150 mg, 1 mg to 100 mg, 1 mg to 75 mg, 1 mg to 50 mg, 1 mg to 25 mg, 100 mg, 50 mg or 25 mg.
34. The in a concentration selected from use any of embodiments 26-33, wherein hemoglobin and/or red blood cell (RBC) count increases relative to baseline of a subject by at least 5%, 10%, 15%, 20%, 25%, 30%, 35%, 40% w, 45% and 50%.
35. The use of any embodiments 26-33, wherein the concentration of Desidustat or a pharmaceutically acceptable salt thereof in parenteral pharmaceutical composition is selected from 1 mg/mL to 250 mg/mL, 1 mg/mL to 200 mg/mL, 1 mg/mL to 150 mg/mL, 1 mg/mL to 125 mg/mL, 1 mg/mL to 100 mg/mL, 1 mg/mL to 75 mg/mL, 1 mg/mL to 50 mg/mL, 1 mg/mL to 25 mg/mL, and 1 mg/mL to 10 mg/mL and the concentration of Desidustat or a pharmaceutically acceptable salt thereof in parenteral pharmaceutical composition is selected from 1% w/w to 90% w/w, 1% w/w to 80% w/w, 1% w/w to 70% w/w, 1% w/w to 60% w/w, 1% w/w to 50% w/w, 1% w/w to 40% w/w, 1% w/w to 30% w/w, 1% w/w to 20% w/w or 1% w/w to 10% w/w.
36. The use of any of embodiments 28-35, wherein the oral pharmaceutical composition comprises solubilizers, diluents or fillers, disintegrants, binder, lubricants, glidants, film forming agents, plasticizers, opacifier, solvents, or a mixture thereof.
37. The use of embodiment 36, wherein the diluent or filler is selected from starch and its processed and co-processed derivatives, saccharides, di saccharides, sucrose, lactose, polysaccharides, cellulose, cellulose ethers, cellulose acetate, hydroxypropyl cellulose, sugar alcohols, xylitol, sorbitol, maltitol, lactitol, microcrystalline cellulose, magnesium or calcium or sodium carbonate, lactose, lactose monohydrate, di-calcium phosphate, compressible sugars, di-basic calcium phosphate dihydrate, mannitol lactose anhydrous, magnesium oxide, maltodextrin, maltose, pullulan, sodium alginate, sodium bicarbonate, calcium silicate, calcium sulphate, cell and tribasic calcium phosphate or suitable combinations thereof, the disintegrant is selected from maize starch, sodium starch glycolate, croscarmellose sodium, crospovidone, microcrystalline cellulose, modified corn starch, sodium carboxymethyl starch, povidone, pregelatinized starch, agar, carboxymethyl cellulose calcium or sodium, colloidal silicon dioxide, chitosan, docusate sodium, hydroxyl propyl cellulose, magnesium aluminium silicate, maltose, methyl cellulose, polacrilin potassium, and alginic acid or suitable combinations thereof, the binder is selected from chitosan, hydrogenated castor oil, sodium alginate, carbomers, cellulose acetate phthalate, povidone, sugar, hydroxypropylmethyl-cellulose, hydroxypropylcellulose, starch, alginic acid, pregelatinized starch, acacia, tragakanth, ethylcellulose, acrylic and methacrylic acid co polymers or suitable combinations thereof; the lubricant is selected from magnesium stearate, stearic acid, silica, fats, zinc or sucrose or sodium or calcium stearate, castor oil, hydrogenated castor oil. Polyethylene glycol and its derivatives, sodium stearyl fumarate, talc, or fatty acids including lauric acid, oleic acid, glyceryl behenate, glyceryl monostearate, and C/CIO fatty acid or suitable combinations thereof, the glidant is selected from colloidal silicon dioxide, talc, fumed silica, starch, starch derivatives, and bentonite or suitable combinations thereof, film forming agent is selected from hydroxypropyl methylcellulose, methylcellulose, ethylcellulose, hydroxypropyl cellulose, povidone, polydextrose, lactose, maltodextrin, acrylic polymer or suitable combinations thereof, the plasticizers is selected from polyols like polyethylene glycols PEG, propylene glycol, glycerol (glycerin), organic esters like phthalate esters (diethyl, dibutyl), dibutyl sebacete, citrate esters (triethyl, acetyl triethyl, acetyl tributyl), triacetin, Oils/glycerides like castor oil; acetylated monoglycerides, fractionated coconut oil or suitable combinations thereof, the opacifier is selected from titanium dioxide, talc, sunset yellow, Tartrazine, Erythrosine, iron oxide yellow, red and black, Carmine, Anthocyanins. Allura Red AC, Allura Red AC aluminum lake, Indigotine, Indigotine aluminum lake or suitable combinations thereof; solvents is selected from water, acetone, chloroform, dichlorome thane, ethyl alcohol, ethyl acetate, methyl alcohol, isopropyl alcohol, N,N-dimethyl formamide and combinations thereof.
38. The method of any of embodiments 13-25, further comprises administration of an additional suitable therapeutic agent to the subject.
39. The method of embodiment 38, wherein the additional suitable therapeutic agent is selected from Neupogen (Filgrastim), Neulasta (Pegfilgrasim), Leukine (Sargramostim), and Nplate (Romiplostim).
   Parenteral composition of Desidustat

### Example 1 and 2

| | |
|---|---|
| Example 1 | Example 2 |

| Ingredients | % w/v | mg/mL | % w/v | mg/mL |
|---|---|---|---|---|
| Desidustat | 5.000 | 50.000 | 10.000 | 100.000 |
| Glycerine | 1.250 | 12.500 | 0.500 | 5.000 |
| Sodium | 0.654 | 6.540 | 1.320 | 13.200 |
| hydroxide | | | | |
| Water for | q.s. | q.s. to 1 | q.s. | q.s. to 1 mL |
| injection | | mL | | |

### Example 3 and 4

| | Example 3 | | Example 4 | |
|---|---|---|---|---|
| Ingredients | % w/v | mg/mL | % w/v | mg/mL |
| Desidustat | 5.000 | 50.000 | 10.000 | 100.000 |
| Sodium | 0.450 | 4.500 | 0.150 | 1.500 |
| Chloride | | | | |
| Sodium | 0.654 | 6.540 | 1.320 | 13.200 |
| hydroxide | | | | |
| Water for | q.s. | q.s. to 1 mL | q.s. | q.s. to 1 mL |
| injection | | | | |

### Example 5 and 6

| | Example 5 | | Example 6 | |
|---|---|---|---|---|
| Ingredients | % w/V | mg/mL | % w/v | mg/mL |
| Desidustat | 4.000 | 40.000 | 2.500 | 25.000 |
| Glycerine | 1.250 | 12.500 | 1.250 | 12.500 |
| Sodium | — | — | 0.544 | 5.440 |
| Chloride | | | | |
| Sodium | 0.544 | 5.440 | 0.175 | 1.750 |
| hydroxide | | | | |
| Water for | q.s. | q.s. to 1 mL | q.s. | q.s. to 1 mL |
| injection | | | | |

### Example 7 and 8

| | |
|---|---|
| Example 7 | Example 8 |

| Ingredients | % w/v | mg/mL | % w/v | mg/mL |
|---|---|---|---|---|
| Desidustat | 4.000 | 40.000 | 4.000 | 40.000 |
| Tromethamine | 1.652 | 16.520 | — | — |
| Meglumine | — | — | 2.659 | 26.550 |
| Water for | q.s. | q.s. to 1 | q.s. | q.s. to 1 mL |
| injection | | mL | | |

Parenteral composition of Roxadustat

### Example 9 and 10

| | |
|---|---|
| Example 9 | Example 10 |

| Ingredients | % w/v | mg/mL | % w/V | mg/mL |
|---|---|---|---|---|
| Roxadustat | 5.000 | 50.000 | 10.000 | 100.000 |
| Glycerine | 1.250 | 12.500 | 0.500 | 5.000 |
| Sodium | 0.654 | 6.540 | 1.320 | 13.200 |
| hydroxide | | | | |
| Water for | q.s. | q.s. to 1 | q.s. | q.s. to 1 mL |
| injection | | mL | | |

### Example 11 and 12

| | Example 11 | | Example 12 | |
|---|---|---|---|---|
| Ingredients | % w/v | mg/mL | % w/v | mg/mL |
| Roxadustat | 5.000 | 50.000 | 10.000 | 100.000 |
| Sodium Chloride | 0.450 | 4.500 | 0.150 | 1.500 |
| Sodium | 0.654 | 6.540 | 1.320 | 13.200 |
| hydroxide | | | | |
| Water for | q.s. | q.s. to 1 | q.s. | q.s. to 1 mL |
| injection | | mL | | |

TABLE 1
Stability data of Example 1

| | | | | 1 Month | 3 Month | 3 Month |
|---|---|---|---|---|---|---|
| Sr. | | | | (40 ± 2° C./ | (40 ± 2° C./ | (25 ± 2° C./ |
| No. | Tests | | Initial | 75 ± 5% RH) | 75 ± 5% RH) | 60 ± 5% RH) |
| 1 | Description | | * | * | * | * |
| 2 | Assay of | | 101.2% | 101.8% | 101.3% | 100.7% |
| | Desidustat (%) | | | | | |
| 3 | Related | DES-7 | 0.06% | 0.07% | 0.07% | 0.07% |
| | substances | Butene | | | | |
| | (%) (By | analog | | | | |
| | HPLC) | Any | 0.01% | ND | ND | ND |
| | | unspecified | | | | |
| | | degradation | | | | |
| | | product | | | | |
| | | Total | 0.11% | 0.06% | 0.07% | 0.07% |
| | | degradation | | | | |
| | | product | | | | |

| | | | | | |
|---|---|---|---|---|---|
| 4 | pH | 7.5 | 7.6 | 7.5 | 7.6 |
| 5 | % Transmittance | 99.33% | 99.86% | 99.46% | 99.95% |
| | at 650 nm | | | | |
| 6 | Osmolality | 297 | 307 | 302 | 299 |
| | (mOsm/kg) | | | | |
| 7 | APHA color | 88 | 72 | 82 | 83 |
| | test (AU) | | | | |

* Clear light yellow solution free from visible particulate matter,
ND: Not detected

TABLE 2
Stability data of Example 2

| | | | | 1 Month | 3 Month | 3 Month |
|---|---|---|---|---|---|---|
| Sr. | | | | (40 ± 2° C./ | (40 ± 2° C./ | (25 ± 2° C./ |
| No. | Tests | | Initial | 75 ± 5% RH) | 75 ± 5% RH) | 60 ± 5% RH) |
| 1 | Description | | * | * | * | * |
| 2 | Assay of | | 102.1% | 101.7% | 99.6% | 99.9% |
| | Desidustat (%) | | | | | |
| 3 | Related | DES-7 | 0.08% | 0.07% | 0.07% | 0.07% |
| | substances | Butene | | | | |
| | (%) (By | analog | | | | |
| | HPLC) | Any | ND | ND | ND | ND |
| | | unspecified | | | | |
| | | degradation | | | | |
| | | product | | | | |
| | | Total | 0.08% | 0.07% | 0.07% | 0.07% |
| | | degradation | | | | |
| | | product: | | | | |

| | | | | | |
|---|---|---|---|---|---|
| 4 | pH | 7.6 | 7.7 | 7.7 | 7.7 |
| 5 | % Transmittance | 99.45% | 99.79% | 99.39% | 99.74% |
| | at 650 nm | | | | |
| 6 | Osmolality | 349 | 353 | 348 | 349 |
| | (mOsm/kg) | | | | |
| 7 | APHA color | 141 | 153 | 165 | 173 |
| | test (AU) | | | | |

* Clear light yellow solution free from visible particulate matter,
ND: Not detected

TABLE 3
Stability data of Example 5

| | | | | 40 ± 2° C./75 ± 5% RH | | | | 25 ± 2° C./60 ± 5% RH | | | |
|---|---|---|---|---|---|---|---|---|---|---|---|
| Sr. | | | | 1 | 2 | 3 | 6 | 1 | 2 | 3 | 6 |
| No. | Tests | | Initial | Month | Months | Months | Months | Month | Months | Months | Months |
| 1 | Description | | * | * | * | * | * | * | * | * | * |
| 2 | Assay of | | 99.4% | 100.2% | 99.1% | 100.8% | 98.7% | 101.4% | 99.6% | 101.0% | 99.1% |
| | Desidustat (%) | | | | | | | | | | |
| 3 | Related | DES-7 | 0.07% | 0.07% | 0.07% | 0.07% | 0.06% | 0.07% | 0.07% | 0.08% | 0.07% |
| | substances | Butene | | | | | | | | | |
| | (%) (By | analog | | | | | | | | | |
| | HPLC) | Any | BQL | ND | ND | ND | ND | ND | ND | ND | ND |
| | | unspecified | | | | | | | | | |
| | | degradation | | | | | | | | | |
| | | product | | | | | | | | | |
| | | Total | 0.07% | 0.07% | 0.07% | 0.07% | 0.06% | 0.07% | 0.07% | 0.08% | 0.07% |
| | | degradation | | | | | | | | | |
| | | product: | | | | | | | | | |

| | | | | | | | | | | |
|---|---|---|---|---|---|---|---|---|---|---|
| 4 | pH | 7.6 | 7.6 | 7.6 | 7.6 | 7.6 | 7.6 | 7.6 | 7.6 | 7.6 |
| 5 | % Transmittance | 99.44% | 99.83% | 100.01% | 99.89% | 99.62% | 100.29% | 100.03% | 99.88% | 99.62% |
| | at 650 nm | | | | | | | | | |
| 6 | Osmolality | 274 | 275 | 274 | 272 | 281 | 273 | 273 | 281 | 277 |
| | (mOsm/kg) | | | | | | | | | |
| 7 | APHA color | 75 | 72 | 88 | 81 | 63 | 72 | 79 | 77 | 57 |
| | test (AU) | | | | | | | | | |

* Clear, light yellow solution free from visible particulate matter,
BQL: Below quantification limit,
ND: Note detected

TABLE 4
Stability data of Example 6

| | | | | 1 Month | 3 Month | 1 Month | 3 Month |
|---|---|---|---|---|---|---|---|
| Sr. | | | | (40 ± 2° C./ | (40 ± 2° C./ | (25 ± 2° C./ | (25 ± 2° C./ |
| No. | Tests | | Initial | 75 ± 5% RH) | 75 ± 5% RH) | 60 ± 5% RH) | 60 ± 5% RH) |
| 1 | Description | | * | * | * | * | * |
| 2 | Assay of | | 101.5% | 100.3% | 100.1% | 100.6% | 100.9% |
| | Desidustat (%) | | | | | | |
| 3 | Related | DES-7 | 0.08% | 0.07% | 0.07% | 0.08% | 0.07% |
| | substances | Butene | | | | | |
| | (%) (By | analog | | | | | |
| | HPLC) | Any | ND | ND | BQL | ND | ND |
| | | unspecified | | | | | |
| | | degradation | | | | | |
| | | product | | | | | |
| | | Total | 0.08% | 0.07% | 0.07% | 0.08% | 0.07% |
| | | degradation | | | | | |
| | | product: | | | | | |

| | | | | | | |
|---|---|---|---|---|---|---|
| 4 | pH | 7.6 | 7.5 | 7.6 | 7.6 | 7.6 |
| 5 | % Transmittance | 99.68% | 99.75% | 99.61% | 99.95% | 99.91% |
| | at 650 nm | | | | | |
| 6 | Osmolality | 290 | 292 | 293 | 295 | 292 |
| | (mOsm/kg) | | | | | |
| 7 | APHA color | 66 | 64 | 54 | 58 | 42 |
| | test (AU) | | | | | |

* Clear colorless solution free from visible particulate matter,
BQL: Below quantification limit,
ND: Note detected

(1-but-3-en-lyloxy)4-hydroxy-2-oxo-1,2-dihydroquinoline-3-carbonyl)glycine is DES-7 Butene analog.

Based on the stability data obtained, pharmaceutical composition of examples 1-8 stored under 25±2° C./60±5% RH and 40±2° C./75±5% RH conditions for up to 6 months, all tested parameters remained within the specified limits. No significant changes were observed in assay, impurity profile, or physical characteristics. The results demonstrate consistent stability across all examples.

### Oral Composition of Desidustat

### Example 13

| Sr. No. | Name of Ingredient | % w/w |
|---|---|---|
| 1. | Desidustat | 40.00 |
| 2. | Microcrystalline Cellulose | 44.00 |
| 3. | Croscarmellose Sodium | 5.00 |
| 4. | Lactose Monohydrate | 5.00 |
| 5. | Hypromellose | 4.00 |
| 6. | Talc | 1.00 |
| 7. | Magnesium stearate | 1.00 |
| 8. | Total | 100.00 |

Example 13 was prepared as per method disclosed in WO2021186356.

### Example 14

### Study Design and Treatment

### Effect of Desidustat Treatment in Radiation Induced Toxicity

Male C57BL/6J (6-8 weeks old) were used in the study. Mice were exposure to ⁶⁰Co-γ rays 30 Gy/min for 10 sec on day 0. Next day, mice were given either vehicle or Desidustat (7.5 mg/kg, Parenteral (prepared as per example 1), or 15 mg/kg, PO, or 30 mg/kg, PO, alternate day) or Roxadustat (15 mg/kg, PO, alternate day) for next 14 days. At the end of treatment period, whole blood samples were processed for hemoglobin, and RBC.

### Effect of Desidustat Treatment on Cyclophosphamide-Induced Bone Marrow Toxicity

Cyclophosphamide induces myelosuppression, leading to the suppression of all nucleated cells in bone marrow by inhibiting DNA replication and directly damaging hematopoietic cells, which reduces their ability to produce red blood cells, white blood cells, and platelets. This leads to decrease in RBC, WBC and platelets in peripheral blood after cyclophosphamide treatment [8-9]. The bone marrow cell type suppression leading to decreased in peripheral cell type by cyclophosphamide is similar to gamma radiation induced depletion of cell type in bone marrow and peripheral blood [8, 10].

Male C57BL/6J mice (6-8 weeks old) were given Cyclophosphamide (100 mg/kg, IP) daily for 4 days. Next day, mice were given either vehicle or Desidustat (7.5 mg/kg, Parenteral, or 15 mg/kg, PO alternate day) or Roxadustat (15 mg/kg, PO, alternate day) treatment for next 14 days. At the end of treatment period, whole blood samples were processed for hemoglobin, and RBC.

### Results

### Effect of Desidustat Treatment on Radiation-Induced Toxicity

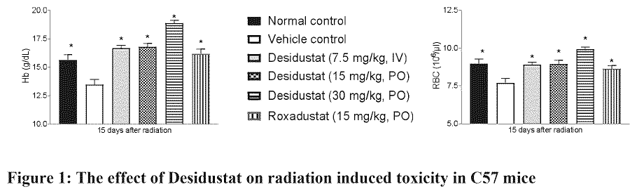

Parenteral treatment of Desidustat at 7.5 mg/kg, significantly increased hemoglobin and RBC by 23.8±1.8 and 15.5±2.1% against vehicle control. Oral treatment of Desidustat at 15 mg/kg significantly increased hemoglobin and RBC by 24.7±2.4 and 16.2±3.2% against vehicle control. Desidustat treatment (30 mg/kg, PO) significantly increased hemoglobin and RBC by 40.3±2.0 and 28.8±1.7% against vehicle control. Roxadustat treatment increased hemoglobin and RBC by 20.3±3.1 and 12.1±2.7% against vehicle control (FIG. 1).

| Sr. | | | Red Blood |
|---|---|---|---|
| No. | Compound | Hemoglobin | Cells (RBC) |
| 1 | Desidustat (7.5 mg/kg, Parenteral) | 23.8 ± 1.8% | 15.5 ± 2.1% |
| 2 | Desidustat (15 mg/kg, Oral) | 24.7 ± 2.4% | 16.2 ± 3.2% |
| 3 | Desidustat (30 mg/kg, Oral) | 40.3 ± 2.0% | 28.8 ± 1.7% |
| 4 | Roxadustat (15 mg/kg, Oral) | 20.3 ± 3.1% | 12.1 ± 2.7% |

### Effect of Desidustat Treatment on Cyclophosphamide-Induced Bone Marrow Toxicity

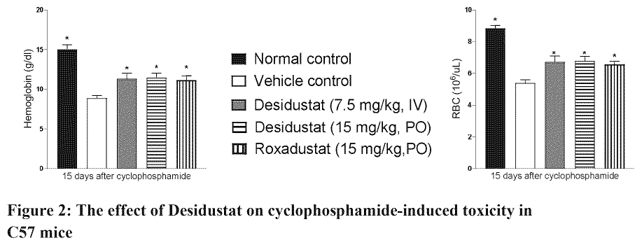

Desidustat treatment by Parenteral route (7.5 mg/kg) significantly increased hemoglobin and RBC by 27.6±7.6 and 24.8±7.0% against vehicle control. Desidustat treatment by oral route (15 mg/kg) significantly increased hemoglobin and RBC by 29.1±6.4 and 26.0±5.2% against vehicle control. Roxadustat treatment increased hemoglobin and RBC by 25.3±6.1 and 22.0±3.4% against vehicle control (FIG. 2).

| Sr. | | | Red Blood |
|---|---|---|---|
| No. | Compound | Hemoglobin | Cells (RBC) |
| 1 | Desidustat (7.5 mg/kg, Parenteral) | 27.6 ± 7.6% | 24.8 ± 7.0% |
| 2 | Desidustat (15 mg/kg, Oral) | 29.1 ± 6.4% | 26.0 ± 5.2% |
| 3 | Roxadustat (15 mg/kg, Oral) | 25.3 ± 6.1% | 22.0 ± 3.4% |

### REFERENCES

1. Acosta, Robert, and Steven J. Warrington. “Radiation Syndrome.” (2017).
2. Bennett C L, Georgantopoulos P, Gale R P, Knopf K, Hrushesky W J, Nabhan C, Armitage J O. United States' regulatory approved pharmacotherapies for nuclear reactor explosions and anthrax-associated bioterrorism. Expert Opin Drug Saf 2023 Jul.-Dec. 22(9):783-788.
3. Joharapurkar A A, Pandya V B, Patel V J, Desai R C, Jain M R. Prolyl Hydroxylase Inhibitors: A Breakthrough in the Therapy of Anemia Associated with Chronic Diseases. J Med Chem. 2018 Aug. 23; 61(16):6964-6982.
4. Tang Y Y, Wang D C, Wang Y Q, Huang A F, Xu W D. Emerging role of hypoxia-inducible factor-1α in inflammatory autoimmune diseases: A comprehensive review. Frontiers in Immunology. 2023 Jan. 25; 13:1073971.
5. Li H S, Zhou Y N, Li L, Li S F, Long D, Chen X L, Zhang J B, Feng L, Li Y P. HIF-la protects against oxidative stress by directly targeting mitochondria. Redox Biol. 2019 Jul. 25: 101109.
6. Jain M R, Joharapurkar A A, Pandya V, Patel V, Joshi J, Kshirsagar S, Patel K, Patel P R, Desai R C. Pharmacological Characterization of ZYAN1, a Novel Prolyl Hydroxylase Inhibitor for the Treatment of Anemia. Drug Res (Stuttg). 2016 February; 66(2):107-12.
7. Joharapurkar A A, Patel V J, Kshirsagar S G, Patel M S, Savsani H H, Jain M R. Prolyl hydroxylase inhibitor desidustat protects against acute and chronic kidney injury by reducing inflammatory cytokines and oxidative stress. Drug Dev Res. 2021 September; 82(6):852-860.
8. Iqubal A, Syed M A, Haque M M, Najmi A K, Ali J, Haque S E. Effect of nerolidol on cyclophosphamide-induced bone marrow and hematologic toxicity in Swiss albino mice. Experimental Hematology. 2020 Feb. 1; 82:24-32.
9. Blankenberg F G, Naumovski L, Tait J F, Post A M, Strauss H W. Imaging cyclophosphamide-induced intramedullary apoptosis in rats using 99mTc-radiolabeled annexin V. Journal of Nuclear Medicine. 2001 Feb. 1; 42(2):309-16.
10. Gul O V, Sengul A, Demir H. Effects of radiation at different dose rates on hematologic parameters in rats. Journal of Radiation Research and Applied Sciences. 2024 Jun. 1; 17(2):100873.

We claim:

1. A parenteral pharmaceutical composition comprising desidustat: 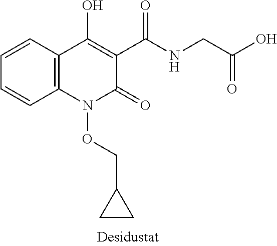 [SMILES: O=C(O)CNC(=O)c1c(O)c2ccccc2n(OCC2CC2)c1=O]
or a pharmaceutically acceptable salt thereof.

2. The composition of claim 1, wherein the desidustat is present at a concentration of 1 mg/mL to 125 mg/mL.

3. The composition of claim 2, wherein the concentration of the desidustat is 15 mg/mL to 35 mg/mL.

4. The composition of claim 2, wherein the concentration of the desidustat is 30 mg/mL to 50 mg/mL.

5. The composition of claim 2, wherein the concentration of the desidustat is 40 mg/mL to 60 mg/mL.

6. The composition of claim 2, wherein the concentration of the desidustat is 90 mg/mL to 110 mg/mL.

7. The composition of claim 2, wherein the concentration of the desidustat is 25 mg/mL.

8. The composition of claim 2, wherein the concentration of the desidustat is 40 mg/mL.

9. The composition of claim 2, wherein the concentration of the desidustat is 50 mg/mL.

10. The composition of claim 2, wherein the concentration of the desidustat is 90 mg/mL.

11. The composition of claim 2, further comprising an isotonic agent/cosolvent.

12. The composition of claim 11, wherein the isotonic agent/cosolvent is selected from glycerine, sodium chloride, and a mixture thereof.

13. The composition of claim 12, wherein the isotonic agent/cosolvent is glycerine.

14. The composition of claim 13, wherein the isotonic agent/cosolvent is present at a concentration of 1% to 1.5% (w/v).

15. The composition of claim 14, wherein the concentration of the isotonic agent/cosolvent is 1.25% (w/v).

16. The composition of claim 2, further comprising an alkalizing agent.

17. The composition of claim 16, wherein the alkalizing agent is selected from sodium hydroxide, meglumine, tromethamine, and a mixture thereof.

18. The composition of claim 16, wherein the alkalizing agent is sodium hydroxide.

19. The composition of claim 18, wherein isotonic alkalizing agent is present at a concentration of 0.5% to 1% (w/v).

20. The composition of claim 19, wherein the concentration of the isotonic alkalizing agent is 0.65% (w/v).

21. The composition of claim 2, further comprising glycerine and sodium hydroxide.

22. The composition of claim 21, wherein the isotonic agent/cosolvent is present at a concentration of 1.25% (w/v) and the sodium hydroxide is present at a concentration of 0.65% (w/v).

23. A composition of claim 1, wherein the composition is as described in any one of Examples 1 to 8.

24. A method of treating radiation induced toxicity in a subject in need thereof, the method comprising administering to the subject a therapeutically effective amount of the pharmaceutical composition of claim 1.

25. The method of claim 24, wherein the radiation induced toxicity is acute radiation syndrome.

26. The method of claim 25, wherein the acute radiation syndrome is hematopoietic acute radiation syndrome (H-ARS).

27. The method of claim 26, wherein 1 mg to 25 mg of the desidustat or a pharmaceutically acceptable salt thereof is administered to the subject.

28. The method of claim 27, wherein the method increases hemoglobin and/or red blood cell (RBC) count relative to baseline of a subject by at least 5% (w/w), 10% (w/w), or 15% (w/w).

29. The method of claim 24, further comprising administering an additional therapeutic agent to the subject.

30. The method of claim 29, wherein the additional therapeutic agent is selected from Neupogen (filgrastim), Neulasta (pegfilgrastim), Leukine (sargramostim), and Nplate (romiplostim).
